(19) United States
(12) Patent Application Publication
(10) Pub. No.: US 2006/0263343 A1
(43) Pub. Date: Nov. 23, 2006

(54) Molecular dissection of cellular responses to alloantigen or autoantigen in graft rejection and autoimmune disease

(76) Inventors: Norma S. Kenyon, Miami, FL (US); Cynthia M. Healy, Miami, FL (US); Steven K. Koester, Pembroke Pines, FL (US); Xiumin Xu, Miami, FL (US)

Correspondence Address: NIXON & VANDERHYE, PC, 901 NORTH GLEBE ROAD, 11TH FLOOR, ARLINGTON, VA, 22203, US

(21) Appl. No.: 11/433,776

(22) Filed: May 15, 2006

Related U.S. Application Data
(60) Provisional application No. 60/680,503, filed on May 13, 2005.

(51) Int. Cl. C12Q 1/68; G01N 33/567; A61K 48/00; A61K 39/395; A61K 31/573; A61K 31/7048; A61K 31/4745; G01N 33/53; A61K 39/00

(52) U.S. Cl. 424/93.21; 424/184.1; 435/6; 435/7.2; 424/155.1; 514/28; 514/171

(57) ABSTRACT

An antigen-specific T-cell response to alloantigen, tissue-specific antigen (e.g., islet antigen or other autoantigens involved in autoimmune disease), or self (or host) antigen is detected at an early stage of graft rejection or recurrent autoimmunity. An increase in cytotoxic lymphocyte gene (CLG) expression in peripheral blood is a risk factor for development of deleterious immune responses, which may be confirmed by functional assays. For example, the distinction between production of regulatory or inflammatory cytokines by T cells may dissect the type of immune response which is being induced: the survival of transplanted islet cells used to treat type 1 diabetes may be monitored, loss of the transplant by graft rejection (i.e., an alloantigen target) may be distinguished from autoimmune disease (i.e., a self or host antigen target).

### CROSS-REFERENCE TO RELATED APPLICATION

This application claims the benefit of provisional U.S. Appln. No. 60/680,503, filed May 13, 2005.

### BACKGROUND OF THE INVENTION

This invention relates to early detection of antigen-specific T-cell responses to alloantigen, tissue-specific antigen (e.g., islet antigen or other autoantigens involved in autoimmune disease), or self (or host) antigen. An increase in expression of granzyme B, perforin, Fas ligand, or any combination thereof in peripheral blood is a risk factor for development of deleterious immune responses to transplanted or host cells, which may be confirmed by functional assays of antigen-specific T cells.

In the prior art, characterization of T cells in patients who are recipients of allografts or those afflicted with autoimmune disease have led to conflicting reports. The determination of immunophenotype by flow cytometry has generally focused on expression of activation markers, and more recently, on intracellular cytokine profile. It has been postulated that the lack of consistency in rejecting vs. clinically stable patients or in patients at different stages of autoimmune disease was due to a lack of relevance of the measurements made of peripheral blood cells to what is happening in target tissue. Alternatively, in patients with autoimmune disease, the assays may not capture changes in immune status that occur with progression of disease. An additional complicating factor is the use of nonspecific stimuli to activate cells, as is routinely done in order to increase the number of putative autoantigen-specific T cells that can be detected.

Clearly, one of the drawbacks of flow cytometry-based methods alone is the inability to effectively detect the functional status of rare event, antigen-specific T cells in peripheral blood without initially expanding clones of T cell clones of interest by antigen-specific or nonspecific stimulation. While nonspecific stimulation might increase the number of antigen-specific T cells, the remaining cells that are not the clones of interest will also be expanded. The end result could be further dilution of the antigen-specific T cell population of interest and an inability to accurately detect these cells, unless the starting T cell clones of interest were frequent enough to preclude complications. With tetramer technology, for example, this is addressed to some extent by combining both antigen-specific and nonspecific T-cell activation techniques. Furthermore, nonspecific activation in vitro can lead to changes in T-cell functional status that are not reflective of the situation in vivo. Improved analysis of the functional status of rare event, antigen-specific T cells is needed to eliminate such variables. In addition, the logistical issues associated with patient blood sampling, including anemia, inconvenience to the patient, limited investigator resources, and sample deterioration due to shipping necessitate that clinical trials are designed to obtain blood at relatively infrequent, predetermined time points (e.g., every three months), yet each individual will progress to graft loss or later disease stages in their own time frame. This means that traditional, predetermined periodic blood collection times will only occasionally lead to a positive finding that host or graft tissue is about to be or is being destroyed. This is further complicated by the fact that most of the immune events will take place within the affected tissue, and detection of rare event cells in peripheral blood will only occur in cases where the cells are captured as they migrate to the target tissue site. Alternatively, cells could be detected in the final stages of tissue destruction in which cells may reenter peripheral blood when tissue destruction is advanced. Our findings (i.e., CLG elevation present in the absence of clinical symptoms and after graft loss) support this contention.

The present invention provides processes that determine the functional status of antigen-specific T cells (reactive against alloantigen, autoantigen, or self antigen targets) in peripheral blood after first determining, in an antigen nonspecific fashion, that rare event, activated or effector T cells are present in peripheral blood. Our previously published results demonstrated that analysis of cytotoxic lymphocyte gene (CLG) expression by RT-PCR enables detection, and possibly, prediction of rejection in islet recipients. Increased CLG expression occurred several weeks prior to the onset of clinical symptoms of graft rejection; equally as important, at the time clinical symptoms occurred (e.g., increased blood glucose levels, increased insulin requirements), CLG expression was negative. Unlike solid organ transplantation, in which chemical markers of rejection allow for therapeutic manipulation of the host, with subsequent graft rescue, it has not been clinically possible to reverse islet allograft rejection. This is most likely due to the fact that most of the islet mass has already been lost by the time clinical symptoms occur.

These data provide a clue as to why results from the analysis of peripheral blood lymphocytes (PBL) may not reproducibly correlate with graft status. Current methods for PBL analysis, such as flow cytometry-based analysis for the expression of cell surface and intracellular markers, or the use of ELISPOT, MLR, tetramers, or other techniques to detect antigen-specific T cells may not be sensitive enough to detect these rare event cells. Theoretically, once lymphocytes have homed to the graft site, expansion of the destructive clone occurs at the site of the transplant and such reactive cells are no longer detectable in peripheral blood. This hypothesis is supported by our data demonstrating undetectable CLG expression at the time of clinical evidence of graft dysfunction (return to insulin dependence, unstable glycemic control), followed by a return of increased CLG expression after graft function has clearly deteriorated. See Han et al. Diabetes 53:2281-2290 (2004). Phenotypic analyses of the peripheral blood of islet-transplant hosts treated with steroid-free immune suppression demonstrates that a decrease in all cell subsets occurs in correlation with the decreased white blood cell counts (WBC) found in patients treated with this immunosuppressive regimen. For hosts with stable graft function, these numbers stay decreased; however, those that lose function experience a gradual return of all cell populations to approximately baseline levels, plus an increase in T cells expressing activation markers.

There is no biochemical measure of islet loss other than elevation in blood sugar, return to exogenous insulin administration, or increase in insulin dose (which occurs after a significant mass of islets has been lost) but molecular assays might be sensitive enough to detect activated effector T cells in peripheral blood prior to the onset of hyperglycemia. Prior reports of increased CLG expression and correlation with ongoing renal allograft rejection in mice and humans led us to question whether or not an increase in CLG expression might be predictive of islet rejection. See Strehlau et al. Proc. Natl. Acad. Sci. USA 94:695-700 (1997); Vasconcellos et al. Transplantation 66:562-566 (1998); Sabek et al. Transplantation 74:701-707 (2002). We first demonstrated in NHP, and then in human recipients of islet allografts, that an increase in CLG expression was found to precede the onset of clinical instability by several weeks. See Han et al. Diabetes 51:562-566 (2002) and Han et al. Diabetes 53:2281-2290 (2004). This immune activation was not detected in cell-based assays, which are time consuming, expensive, and require a much larger blood volume than the present invention. The results presented herein show that an increase in CLG expression is an early predictor of immune activation and recognition of alloantigen, autoantigen, or self antigen.

Utilization of increased CLG expression in a molecular assay that signals the need for cellular assays would facilitate monitoring in animal studies and clinical trials of treatments for immune-mediated diseases. Due to the time consuming nature of cellular assays, the need for large blood volumes (generally up to 80 mL blood), and the complexity of such assays, blood samples were not frequently collected (e.g., at three-month intervals) in the prior art. Each subject (i.e., animal model or patient) will, however, progress to graft rejection or recurrent autoimmune disease at a different rate. The critical (informative) cells are rare and they are difficult to detect even when present in the subject's peripheral blood. Moreover, by the time symptoms become evident, the critical cells may already have migrated to the site of the transplant or disease, and thus are absent from the peripheral blood. Molecular assays allow for amplification of signals in rare event cells, require small blood sample volumes (up to 10 mL) and, therefore, patient samples can be collected on a more frequent basis. We show below that an increase in CLG expression is an early predictor that an antigen-specific immune response might have been initiated and more complicated cellular (i.e., functional) assays which involve stimulation with specific antigen may then be performed.

Further objectives and advantages of the invention are described below.

### SUMMARY OF THE INVENTION

An objective of the present invention is to identify risk factors for development of an antigen-specific T-cell response in a subject to one or more alloantigens of a graft (i.e., solid organ transplant, a tissue like pancreatic islets, cells such as stem cells), autoantigens, or self antigens. The subject may be a human patient or an animal disease or transplantation model. It is an advantage that the increase in expression of at least one of granzyme B, perforin, and Fas ligand can be detected before cell or tissue destruction by activated antigen-specific T cells, which mediate graft rejection and autoimmune disease.

In one embodiment of the present invention, samples are obtained from a subject; expression of at least one of the group consisting of granzyme B, perforin, and Fas ligand (i.e., cytotoxic lymphocyte genes) is measured; and, if expression of one or more CLGs is increased, detecting the presence of at least T cells recognizing alloantigen, autoantigen, or self antigen by a functional assay. An alteration in the expression of other genes associated with immune activation and inflammation may also be used as a “molecular flag” indicating that a functional assay to confirm the presence of at least T cells recognizing alloantigen, autoantigen, or self antigen.

Also provided are processes for using combinations of lymphocyte-specific antibodies for diagnosis and making diagnostic kits containing these antibodies. It should be noted, however, that a claim directed to the product is not necessarily limited to these processes unless the particular steps of the process are recited in the product claim. Further aspects of the invention will be apparent to a person skilled in the art from the following description of specific embodiments and the claims, and generalizations thereto.

### BRIEF DESCRIPTION OF THE DRAWINGS

FIG. 1 compares white blood cell count (WBC×10³ cells per μL), subsets by immunophenotyping (cells per μL), and granzyme B (GB) gene expression (mRNA/Actin) % between patients who would eventually experience islet rejection (R) and those who retain stable graft function (S) before immune suppression and islet transplantation (Pre-Txpl). The following peripheral blood mononuclear cell (PBMC) subsets were analyzed by multiparametric flow cytometry: total T cells (CD3/45), memory T cells (CD3/CD45RO) which have previously encountered antigen, naïve T cells (CD3/CD45RA) which have not previously encountered antigen, activated helper T cells (CD3/4) or activated cytotoxic T cells (CD3/8) as assessed by MHC class II (DR) expression, CD69 or CD25, natural killer (NK) cells (CD56/16/3−), and B cells (CD20/40/19). Data are represented as absolute lymphocyte counts, obtained by multiplying the percentage positive cells in the lymphocyte gate by the absolute lymphocyte count. Data were analyzed by mixed model regression (p<0.05 was considered a statistically significant difference).

FIG. 2 shows granzyme B (GB) expression for two representative “rejector” (FIG. 2A) and “stable” (FIG. 2B) patients as a function of time (post-operative day or POD). The data can be dissected into four different phases: clinically stable, no elevation of GB (CS/M−) for both R and S patients; clinically stable, elevation of GB (CS/M+) for the R patient; clinically unstable, GB not detectable (CUS/M−) for the R patient; and clear graft loss, GB reappears (GL/M+) for the R patient, who eventually experienced graft loss. The S patient maintained stable graft function and did not experience the different phases of GB expression, as described for the R patient.

FIG. 3 shows the mean granzyme B (GB) expression of patients that would eventually experience islet rejection (R) and those who retain stable graft function (S) in the various phases of clinically stable (CS), clinically unstable (CUS), or graft loss (GL) for whom the molecular marker is increased (M+) or is not (M−). P value is indicated for the comparison between R and S patients in the indicated phase of treatment; p<0.05 was considered a statistically significant difference.

FIGS. 4 to 7 compare the white blood cell count (WBC×10³ cells per μL), subsets by immunophenotyping (cells per μL), and granzyme B (GB) gene expression between patients who would eventually experience islet rejection (R) and those who retain stable graft function (S). The following subsets of peripheral blood mononuclear cells (PBMC) are defined by their markers indicated in parentheses: total T cells (CD3/45), memory T cells (CD3/RO or CD45RO) which have previously encountered antigen, naïve T cells (CD3/RA or CD45RA) which have not previously encountered antigen, activated helper T cells (CD3/4) or activated cytotoxic T cells (CD3/8) as assessed by MHC class II (DR) expression, CD69 or CD25, natural killer (NK) cells (CD56/16/3−), and B cells (CD20/40/19). Data are represented as absolute lymphocyte counts, obtained by multiplying the percentage positive cells in the lymphocyte gate by the absolute lymphocyte count. Clinically stable patients for whom the molecular flag was not increased (CS/M−, FIG. 4); clinically stable patients for whom the molecular flag was increased (CS/M+, FIG. 5); clinically unstable, hyperglycemic patients for whom the molecular flag was not increased (CUS/M−; FIG. 6); and hyperglycemic patients for whom significant graft function was lost, insulin administration was resumed and GB expression was increased (GL/M+; FIG. 7) were analyzed separately by mixed model regression (p<0.05 was considered a statistically significant difference).

FIG. 8 shows data from patients who eventually experience rejection (R) and in the various phases of clinically stable (CS), clinically unstable (CUS), or graft loss (GL) for whom the molecular marker is increased (M+) or is not (M−) analyzed to determine if statistically significant differences could be observed in nonstimulated whole blood as patients progress along the pathway shown in FIG. 2A. P less than 0.05 was considered a statistically significant difference.

FIGS. 9-25 show the mean granzyme B (GB) expression of patients that would eventually experience islet rejection (R) and those who retain stable graft function (S) in the various phases of clinically stable (CS), clinically unstable (CUS), or graft loss (GL) for whom the molecular marker is increased (M+) or is not (M−). P value is indicated for the comparison between R and S patients in the indicated phase of treatment; p<0.05 was considered a statistically significant difference.

FIGS. 26-29 show flow cytometric analyses of human peripheral blood mononuclear cells (PBMC) from a long-term islet transplant patient (post-operative day or POD 1040), who received islet infusions from multiple donors: unstimulated PBMC (FIG. 26), nonspecific stimulation with dextran crosslinked anti-CD3 plus anti-CD28 (FIG. 27), antigen-specific stimulation with donor 1369 cells (FIG. 28), and antigen-specific stimulation with donor 1461 cells (FIG. 29). Cells were gated for CD3 staining (FIGS. 26A, 27A, 28A, 29A) or CD4 staining (FIGS. 26B, 27B, 28B, 29B) and then further analyzed for staining of CD69, interferon gamma (IFNγ), and interleukin-10 (IL-10).

FIGS. 30-32 show flow cytometric analyses of human peripheral blood mononuclear cells (PBMC) labeled with carboxyfluorescein diacetate, succinimidyl ester (CFSE): unstimulated PBMC (FIG. 30), nonspecific stimulation of PMBC with dextran crosslinked anti-CD3 plus anti-CD28 (FIG. 31), and alloantigen-specific stimulation of PMBC (FIG. 32). After culturing six days, cells were stained for CD3, CD4, CD8, CD16, or CD19. Plots of CD3 vs. CFSE (FIGS. 30A, 31A, 32A); CD4 vs. CFSE (FIGS. 30B, 31B, 32B); CD8 vs. CFSE (FIGS. 30C, 31C, 32C); CD16 vs. CFSE (FIGS. 30D, 31D, 32D); and CD19 vs. CFSE (FIGS. 30E, 31E, 32E) are shown.

### DESCRIPTION OF SPECIFIC EMBODIMENTS OF THE INVENTION

Cytotoxic lymphocyte gene (CLG, preferably granzyme B) expression may be measured at the level of RNA transcription (e.g., microarray or other hybridization techniques, nuclease protection, primer extension, probe hybridization, RT-PCR) or protein translation (e.g., separation by chromatography or electrophoresis; detection by immunoassay, mass spectrometry, or NMR; techniques such as ELISA, immunofluorescent staining, immunohistochemistry, or Western blotting). An increase of CLG expression may be detected relative to a baseline established during normal health, prior to treatment for disease (e.g., an immunosuppressive regimen), or before transplantation. Or the increase may be detected by comparison between successive samples, among a series of samples taken after the initiation of treatment or after transplantation, or other analysis in which frequent sampling soon after transplantation permits early detection of autoimmunity or graft rejection.

The presence of antigen-specific lymphocytes (especially T cells) or natural killer (NK) cells in a sample may be confirmed by functional assays (e.g., cell proliferation, cytokine production and profiling, ELISPOT, immunophenotyping, limiting dilution analysis, mixed lymphocyte reaction, stimulation with specific antigen, tetramer staining) of cells for (i) antigen specificity (e.g., alloantigen, autoantigen, or self antigen), (ii) major histocompatibility complex (MHC) restriction (e.g., donor or host/recipient MHC), and (iii) activation status of antigen-specific lymphocytes or NK cells. In functional assays, it is preferred that stimulation with specific antigen; MHC-restricted antigen presentation; and/or measuring proliferation of specific lymphocyte subsets, expression of activation markers, cytokine or chemokine production, activity of cytokine or chemokine receptors, changes in gene expression, or any combination thereof be incorporated. Clinical intervention (e.g., steroid-free immune suppression) may be initiated to prevent destruction of target cells by lymphocytes and NK cells detected in the sample.

Examples of solid organs and their cells, as well as other cell and tissue types, which may be transplanted from donor to host (or recipient) include: bone marrow, heart, hepatocytes, kidney, liver, lung, neural cells, pancreas, pancreatic islet cells, and stem cells (hematopoietic, mesenchymal, embryonic, other stem cell types, or stem cell-derived tissue). Graft rejection results in destruction of transplanted tissue. Organ-specific autoimmune diseases include Addison's disease, type 1 diabetes, Graves' disease, and Hashimoto's thyroiditis especially those autoimmune diseases that are of a recurrent or relapsing nature. It is preferred that the initial sample be obtained prior to the initiation of immune intervention, or immune suppression and transplantation.

Samples (e.g., peripheral blood, interstitial fluid, lymph, plasma, serum, other fluids or tissues of the donor or host) obtained from the subject may be in volumes of less than 1 mL, 3 mL, 5 mL, or 10 mL. The patient's finger could be pricked, blood could be dropped on absorbent material for storage, and the dried sample could be subsequently analyzed. Samples obtained from the subject at intervals of at least once a day, once a week, every two weeks, once a month, or every two months. An initial sample may be obtained from the subject within 24 hours, one week, two weeks, three weeks, or one month of transplantation; a pre-sample may be obtained from the subject before immune suppression (i.e., a pre-treatment for transplantation) and/or transplantation.

In one embodiment, graft rejection by the host can be distinguished from recurrence of autoimmunity. In the context of islet transplantation, islet destruction may be mediated by recognition of alloantigen, autoantigen, or a combination thereof. Therefore, it is preferred that the functional capability of antigen activated T cells (e.g., using donor cells to activate alloantigen-specific T cells and peptide pools derived from autoantigens of target tissue to activate autoimmune T cells) be determined by analysis of proliferative capacity, distinction between profiles of regulatory or inflammatory cytokines, expression of intracellular or cell-surface proteins associated with different cell subsets (e.g., regulatory vs. effector cells), and expression of activation markers. Lack of proliferation capacity by antigen activated lymphocytes could indicate that the antigen-specific cells have been deleted from the peripheral blood or that regulatory cells are suppressing such proliferation. Antigen-specific regulatory cells should be detectable by their expression of differentiation markers (e.g., Foxp3, cytokines, chemokines), cell-surface marker expression, and secreted cytokine profile. A regulatory cytokine profile (e.g., IL-10 and/or TGF-beta) or an inflammatory cytokine profile (e.g., IFN-gamma and/or TNF-alpha) should be detectable after stimulation with specific antigen, and the T-cell subset (e.g., CD4+ vs. CD8+) can be determined. At the level of a single cell, production of intracellular cytokines may be distinguished from secreted cytokines: e.g., flow cytometry can detect intracellular cytokines after cell permeabilization and ELISPOT can detect cytokines secreted by the cell.

A functional assay may be comprised of donor or host MHC or tissue antigens, antigen presentation cells (e.g., B cells, dendritic cells, macrophages, or any source of donor tissue), cytokines or growth factors, endogenous or exogenously added antigen (e.g., alloantigen, autoantigen, or self antigen), inhibitory or stimulatory antibodies or soluble mediators, or any combination thereof. At least cells and/or soluble factors (e.g., cytokines or chemokines) of the sample interact with other components of the assay (e.g., by physical contact between cells or receptor-ligand binding) and results are measured. In vitro detection of immune reactions are indicative of the occurrence of autoimmune disease or graft rejection in the host (or recipient) in vivo, or the potential of such deleterious event occurring. It is preferred that detection occur early enough in the disease or rejection process to prevent or ameliorate tissue destruction (e.g., “substantial” tissue destruction may be detected by physiological changes such as hyperglycemia or need for exogenous insulin administration).

A preferred embodiment of the invention includes a functional assay which is multiparametric flow cytometry: multiple antibodies added to an assay which are distinguishable and separable by their different labels, simultaneous binding to cells of the sample or cells isolated therefrom, determining which subset (e.g., CD4+ or CD8+ T cells) are responding to antigenic stimulation (inferring antigen and MHC recognition), whether or not cells have proliferative capacity, whether or not an activation marker is expressed on cells, and whether or not inflammatory or regulatory cytokines are being produced (e.g., effector vs. regulatory T cells).

Another preferred embodiment of the invention includes a functional assay which detects individual antigen-specific T cells with an appropriate tetramer (e.g., fluorescent staining with labeled HLA-peptide tetramers for detection of islet-specific autoantigenic T cells). “Tetramer” technology refers to soluble MHC tetramers that are characterized as HLA class I or II, donor or host HLA allele(s), peptide epitope(s) of the antigen, and optional fluorescent label(s). They may be custom synthesized or obtained commercially as iTAg™ tetramers. In situ detection or quantitation by flow cytometry can be used to characterize antigen-specific T cells. See Bleesing & Fleisher Semin. Hematol. 38:169-178 (2001) and Kita et al. Autoimmune. Rev. 2:43-49 (2003).

Yet another embodiment of the invention is distinguishing inflammation due to infection, drug treatment, surgery, or trauma from immune-mediated tissue destruction (e.g., alloantigen, autoantigen, or self antigen). Therefore, in a preferred embodiment, confounding results of infection, drug-induced inflammatory changes, surgery, or trauma (e.g., an increase in expression of at least one of granzyme B, perforin, and Fas ligand) are distinguished by collection of additional clinical information (e.g., detection of a pathogen, history of other treatments that can cause an inflammatory change) and, ultimately, by measuring appropriate markers (e.g., bacterial, fungal, or viral antigen or reactive antibodies indicative of infection by a pathogen) that are capable of distinguishing immune reactions due to infection from immune reactions due to tissue destruction. It is also possible to stimulate T cells with pools of antigenic peptides derived from various pathogens (e.g., CMV, EBV) and to determine whether there is increased reactivity to pathogen as opposed to donor cells. An increase in granzyme B expression only may be indicative of graft rejection, while increases in perforin and Fas ligand expression without increased granzyme B expression may be indicative of recurrent autoimmune disease, or possibly, chronic as opposed to acute rejection.

The present invention may be practiced as a method to monitor development of an antigen-specific T-cell response to alloantigen, autoantigen, or self antigen against target cells of a subject. Alternatively, a kit to practice that method may be provided. The kit, which is optionally stored and transported in a single package, is comprised of one or more containers of reagents or mixtures thereof with an optional set of instructions to practice the method. The reagents may include: (i) reagents to determine CLG expression (e.g., a nucleic acid primer or probe specific for CLG transcribed RNA or translated protein, an antibody specific for CLG antigen, a label to detect CLG expression, one or more standard(s) and/or control(s) to quantitate the amount of CLG expression, or any combination thereof); (ii) reagents to determine whether T cells recognize alloantigen, autoantigen, or self antigen by a functional assay (e.g., one or more alloantigen(s), autoantigen(s), tissue-specific antigen(s), antigen presenting cells of the donor and/or host HLA class I/class II allele(s), cell dyes or stains, donor or host cells, HLA-peptide tetramer(s) with or without label, antibodies for cytokine(s) or leukocyte antigen(s), an ELISA or ELISPOT plate(s), labeled secondary antibodies, or any combination thereof); (iii) reagents for cell culture (e.g., a collection flask(s) or tube(s), a cytokine(s) or growth factor(s), liquid medium, nonspecific or specific T-cell activators, or any combination thereof) and/or cell separation (e.g., density or velocity centrifugation medium, a centrifugation tube(s), a chromatography column(s) or magnetic bead(s) for affinity purification, antibodies against a cell-surface molecule, or any combination thereof); and/or (iv) reagents for cell analysis (e.g., cell dyes or stains, cell lysis or permeabilization buffers, wash buffer(s), antibody cocktails with four or more different fluorescent labels, data processing and analysis software stored in tangible memory, standard(s) or control(s) for cell size, fluorescent signal, gating, etc.). Exemplary antibodies for the kit may recognize the intracellular, secreted, or cell-surface antigens described below as well as granzyme B, Fas ligand, perforin, CXCR3, NKG2D, and HLA-DR3/DR4. Determining CLG expression and the functional assay may be performed separately or together using the same or different samples.

The terms self, host, and recipient largely overlap and are used depending on context, (i.e., graft rejection or autoimmunity). Similarly, the terms donor, graft, and transplant are largely overlapping. Alloantigens include human leukocyte antigens (HLA) of the donor. Self antigens may include autoantigens of a target tissue or solid organ, as well as recipient HLA. Treatment of diabetic patients by islet transplantation may be studied in both aspects: allograft rejection (e.g., against foreign HLA) and/or autoimmune disease (e.g., against islet-specific antigen).

CLG expression is increased before the onset of clinical symptoms of disease. When there is initial clinical evidence of graft loss, however, CLG expression has returned to baseline and does not appear again until significant loss of islets has occurred. These data suggest that sensitive detection of CLG expression allows us to detect donor-specific T cells that are in the process of homing to the graft after first being activated in the draining nodes of the liver. Alternatively, reactivation of autoantigen-specific T cells is being detected.

An increase in CLG expression was found to be predictive of allograft rejection by taking four nonhuman primates (NHP) with stable graft function and discontinuing immune suppression. Prior to initiating a study of human graft recipients, CLG expression was measured by RT-PCR in peripheral blood samples obtained from patients with long-standing type 1 diabetes who were C-peptide negative (no evidence of residual beta cell function) and on a transplant waiting list. A statistically significant difference in the expression of perforin or Fas ligand between type 1 diabetes patients on the waiting list vs. normal controls was observed. But expression of granzyme B was similar for both groups of patients. Subsequently, peripheral blood samples were taken from islet recipients prior to the initiation of immune suppression and pre-transplant as a baseline. Additional samples were collected periodically post-transplant. In the absence of other reasons (e.g., an upper respiratory infection), an increase in CLG expression above baseline for two or more time points may be considered an early predictor of a developing immune response, despite the absence of clinical symptoms of rejection (i.e., the patient has normal blood glucose levels and remains insulin independent) and a lack of altered reactivity with cellular assays. The increase in expression may be at least two times, five times, or ten times baseline. The sample volume was small, which allows frequent drawing of peripheral blood from post-transplant patients including, if necessary, those having decreased white blood cell count (WBC) and anemia.

It was confirmed that 8/8 patients who experienced clinical graft loss (e.g., hyperglycemia, return to exogenous insulin administration) had an increase in CLG expression in peripheral blood which appeared several weeks prior to the onset of hyperglycemia and reinstitution of insulin therapy. In ⅞ patients, this increase occurred during tapering of immune suppression (IS, n=6) or during a prolonged period of inadequate IS (n=1). For ⅞ patients, sensitization to donor antigens was observed in mixed lymphocyte reactions (MLR), which assays the ability of the recipient's peripheral blood lymphocytes (PBL) to proliferate in response to donor cells. On a longer-term basis, increased perforin and Fas ligand expression appears to correlate with recurrent autoimmunity, thereby suggesting that it might be possible to identify immune associated genes that delineate rejection vs. recurrent autoimmunity in a setting in which a clear difference exists in the study population (patients with type 1 diabetes have significantly lower levels of perforin and Fas ligand expression, and similar granzyme B expression, when compared to controls).

Samples were collected on a more frequent basis for molecular analyses of patient blood, primarily because it was feasible to do so, as the sample volume is small. MLR and flow cytometry assays were done less frequently but, from the data generated, it appears that while cellular assays performed alone are correlative with graft loss, they are not reliably predictive. In addition, each cellular assay measures only one parameter. CLG analysis can show that cytotoxic effector gene expression is increased in whole blood but does not allow one to distinguish whether NK cells or cytotoxic T lymphocytes (CTL) are responsible. Proliferation in MLR does not determine whether CD4+ or CD8+ cells are responding and does not distinguish between proliferation of regulatory cell populations (e.g., CD4+ regulatory T cells that produce cytokines such as IL-10 or TGF-beta) and effector cell populations (e.g., CD4+ effector T cells that produce cytokines such as IFN-gamma or TNF-alpha). Our invention addresses these problems by improving reliability such that autoimmunity or graft rejection can be detected early enough to allow clinical intervention prior to substantial tissue destruction and consequent changes in physiology.

We have also developed methods for determining the proliferative capacity and cytokine profile of specific subsets by multiparametric flow cytometry. Panels of antibodies were developed that enhance our ability to assess the functional status of antigen-specific T cells. As an example, the recipient's peripheral blood mononuclear cells (PBMC) stimulated in mixed lymphocyte culture with donor antigen did not proliferate after the patient had been on steroid-free immune suppression (SFIS) for several weeks. This is could be due to a lack of donor-specific cells or to the presence of regulatory populations. In order to address this, anti-cytokine-specific monoclonal antibodies were included in the panels of antibodies specific for CD4+ and CD8+ T cells, starting with IL-10 and TGF-beta as regulatory cytokines and IFN-gamma and TNF-alpha as inflammatory cytokines. To determine if granzyme B expression by flow cytometry would correlate with molecular expression, antibody to granzyme B was included in CD8 tubes, as were markers that distinguish between memory and effector cytotoxic cells. The panel was expanded with additional anti-cytokine antibodies and to allow analysis of CD8+ T cells. Both resting and stimulated cell populations are assessed: resting whole blood, MLR-stimulated PBMC, and CD3/CD28-stimulated PBMC. Inclusion of potent CD3/CD28 stimulation enables verification that the patient is not over immunosuppressed and MLR stimulation determines whether islet graft loss is due to rejection of the transplant or recurrent autoimmunity. Since major changes in whole blood phenotype occur after graft loss has occurred and it is too late to intervene, identification of molecular profile changes can be used as an early signal to perform the more time-consuming cellular analyses for donor and autoantigen responsiveness. Frequent determination of cellular reactivity by MLR with donor cells or other functional assays may result in early detection of immune activation before effector cells have caused destruction of target tissue.

Antibodies that enable delineation of lymphocyte subsets (e.g., T, B, NK cells) and the activation status of peripheral blood lymphocytes (e.g., whether or not a CD3+/CD4+ T cell is expressing the activation marker CD69), as well as at the same time determining whether or not activated T cells (e.g., CD3+/CD4+/CD69+) can proliferate and produce inflammatory or regulatory cytokines, can be placed in one tube. Each tube would contain fluorochrome-conjugated monoclonal antibodies specific for various lymphocyte subsets and cytokines. Therefore, a panel of such antibodies could enable definition of the functional status of specific T cell subsets (i.e., antigen specificity indirectly achieved by stimulating the cells prior to analysis with alloantigen, autoantigen, or self antigen), including but not limited to CD3+/CD4+ regulatory or inflammatory T cells, CD3+/CD8+ regulatory or effector T cells, effector or naïve B cells, and resting or activated NK cells. A dye carboxyfluorescein diacetate, succinimidyl ester (CFSE) can be used to label cells, which take up the dye by an active process. As each cell divides, the daughter cells have half the original amount of CFSE and this continues with each cell division. It is possible to analyze cells that have divided one or more times using the gating function of a flow cytometer and to determine whether or not it expresses markers of a particular cell subset (e.g., a CD3+/CD4+ T cell), whether or not it expresses markers associated with an effector immune response (e.g., CD69, CD25, HLA-DR, CD45RO, etc. and inflammatory cytokines such as IFN-gamma or TNF-alpha), or whether or not it expresses markers associated with a regulatory immune response (e.g., CD25 bright, IL-7 receptor, Foxp3, etc. and regulatory cytokines such as TGF-beta and IL-10).

In another example, cells could be labeled with CFSE and then stained with monoclonal antibodies specific for at least one marker that identifies the lymphocyte subset, at least one marker that identifies antigens associated with activation, at least one marker distinguishing naïve vs. effector vs. memory cells, and at least one marker distinguishing regulatory vs. effector function. Immunophenotyping panels of four or more fluorochrome-conjugated monoclonal antibodies can be used. Many more different monoclonal antibodies can be used with available technology (i.e., the availability of nonoverlapping fluorochrome labels, multicolor flow cytometry, and software to analyze the data) can be used.

As the number of markers increases, it is easier to identify specific lymphocyte subsets. For example, a CD3+/CD4+/CD25+/Foxp3+ cell that produces TGF-beta in response to donor stimulation would be a regulatory cell and would generally associate with graft stability (such cells may or may not proliferate), whereas a CD3+/CD4+/CD25+ cell that is Foxp3 negative, which proliferates and produces TNF-alpha in response to donor stimulation, would be considered an effector cell that is associated with destruction of the graft. In the preceding example, six different fluorochromes would be required. It is possible to make these determinations with as few as four antibodies per tube, but it would require more tubes to adequately assess the required markers.

Multiparameter analysis by flow cytometry of intracellular cytokine expression in resting or activated (e.g., nonspecific activation with anti-CD3 and anti-CD28 stimulatory antibodies, phorbol esters, ionophores, superantigens, mitogens, or any combination thereof) peripheral blood T cells is possible. Subsequent to nonspecific stimulation, it is especially easy to identify activated T cells, expressing markers such as CD25, HLA-DR, CD69, and CD154, and to observe the intracellular expression of cytokines associated with an effector immune response. In normal cell populations, regulatory cytokines, such as IL-10 and TGF-beta, are not observed after nonspecific stimulation.

Nonspecific activation does not necessarily provide a clear indication of how a patient is responding to a transplant or an autoantigen. Peripheral blood samples of islet recipients were analyzed by flow cytometry: resting whole blood, the recipient's PBL activated by donor cells in MLR, and the recipient's PBL activated with a CD3-CD28 bead-based technology have been assessed for cell surface and intracellular markers. IL-10 (i.e., a regulatory cytokine) expression in response to donor cell stimulation was found in only two of the patients. Some IL-10 was also detectable in their resting whole blood (e.g., activated CD4+/CD69+ T cells) but not IFN-gamma. Upon nonspecific activation (e.g., anti-CD3 dex plus anti-CD28 stimulatory antibodies), however, those cells were diluted out along with their IL-10 expression, thereby demonstrating the critical need for analysis of T cells that have been activated with specific antigen—in this case, donor alloantigen. Activation with donor cells in MLR allows detection of an increase in IL-10 producing (regulatory) cells; a low level of IFN-gamma producing (inflammatory) cells might indicate balance between the two cell populations while a high level of IFN-gamma producing cells might indicate a shift to graft rejection.

In summary, even with a multiparametric approach to assess the functional status of antigen-specific T cells, it has become clear that activation of the immune response at the molecular level is not easily detectable with standard cell-based assays alone. Utilization of a molecular flag to trigger more specific assessments of cellular reactivity should provide a better understanding of a patient's immune status, thereby allowing for early intervention to halt rejection and/or recurrent autoimmunity. The molecular flag may be one or more of the cytotoxic lymphocyte genes, but an alteration in the expression of other genes associated with immune activation and inflammation may also be used.

### EXAMPLES

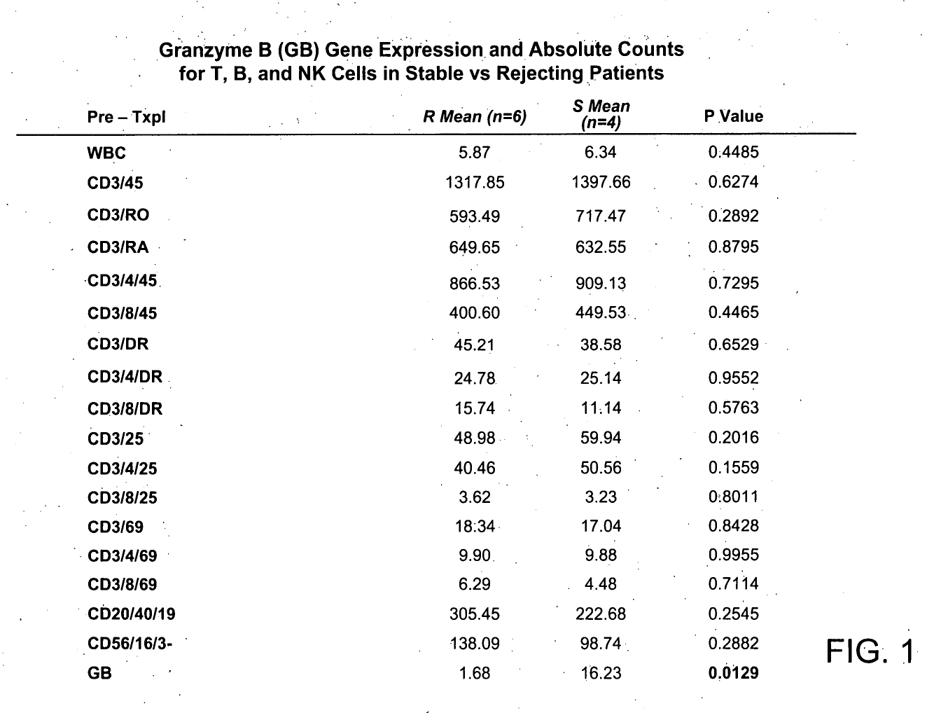

Prior to transplantation, there was no statistically significant difference in WBC between patients who would eventually experience islet rejection and those who retain stable graft function (FIG. 1). Granzyme B (GB) expression was quantitated by real-time polymerase chain reaction (RT-PCR) as described by Han et al. (Diabetes 53:2281-2290, 2004). Flow cytometry was undertaken by addition of 4-color cocktails of monoclonal antibodies, specific for particular human cluster differentiation (CD) antigens, to whole blood followed by a 15 min incubation and processing with a Beckman-Coulter Q-Prep. Analysis was done with an EPICS XL flow cytometer. No statistically significant difference in immunophenotype was observed. Prior to initiation of immune suppression and transplantation, the only statistically significant difference found between patients who would eventually experience islet rejection and those who retain stable graft function was for granzyme B alone. This difference was most likely due to an extremely high pre-transplant, pre-immune suppression GB value for one of the stable patients. But when this exceptionally high, outlying value was eliminated from analysis, the difference was no longer considered significant (p=0.6963).

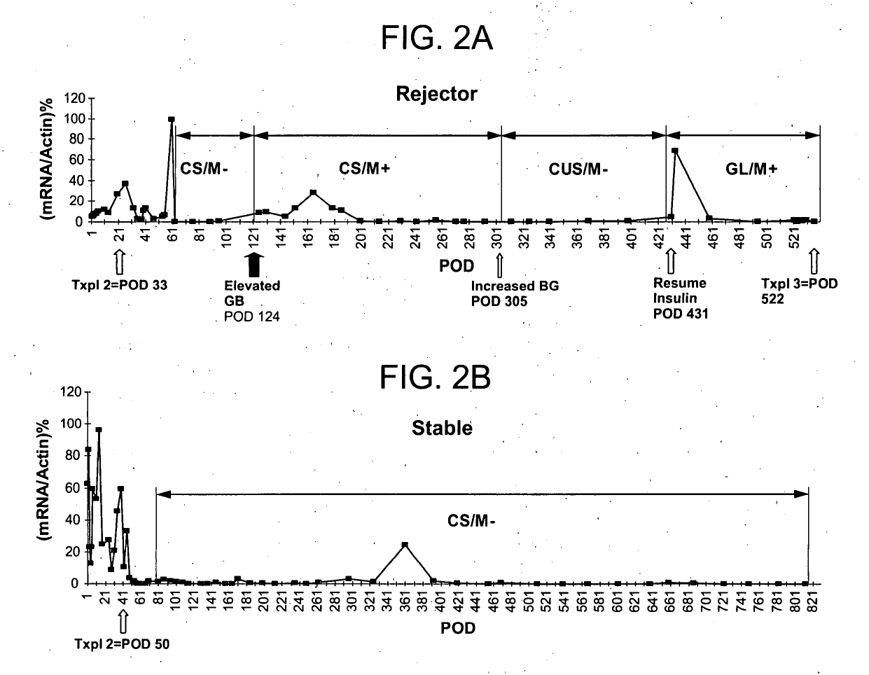

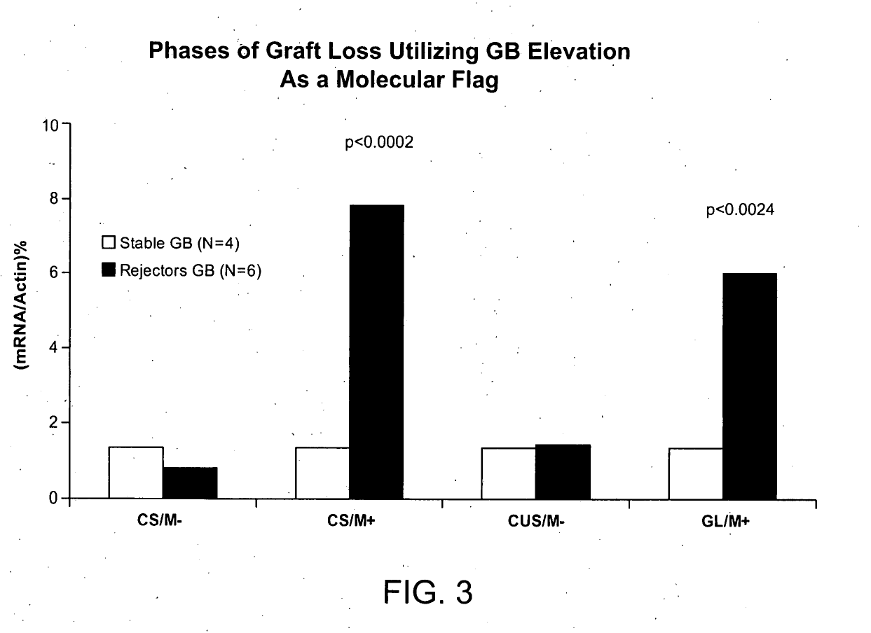

Retrospective analysis of RT-PCR data revealed that, despite normoglycemia and insulin independence, and in the absence of infection in the respiratory tract, an increase in GB expression occurred in 8/8 patients who eventually experienced at least partial islet allograft loss. In the absence of documented clinical infection, no such increase was detected in patients who retained graft function. FIG. 2 dissects the clinical phases of possible graft loss utilizing GB expression as a function of the number of post-operative days for two representative patients. The patient who eventually experienced graft loss was identified as “rejecting” (R) and the patient who maintained stable graft function and did not experience the different phases of GB expression was identified as “stable” (S). Elevation of GB was used as a “molecular flag” that signaled immune activation in the absence of clinical evidence of graft loss by dissecting the flow cytometry data into four different phases (FIG. 3): clinically stable, no elevation of GB (CS/M−) for both R and S patients; clinically stable, elevation of GB (CS/M+) for the R patient; clinically unstable, GB not detectable (CUS/M−) for the R patient; and clear graft loss, GB reappears (GL/M+) for the R patient.

We observed that, despite initiation of immune suppression, most patients experienced elevated GB levels in the first month post-transplant, but in the absence of subsequent transplants, the GB levels returned to baseline without evidence of rejection. Subsequent elevations that occurred in the absence of either additional islet infusions or infections, were correlated with eventual graft loss. For both R and S patients, increases in GB expression after infusion of the graft were observed that eventually returned to baseline. For the R patient, it can be clearly seen that the GB level was elevated at several time points that occurred in the absence of clinical symptoms of islet loss (stable blood glucose, no exogenous insulin, CS/M+). The switch to the clinically unstable phase (i.e., hyperglycemia, still no exogenous insulin) occurred at POD 305 with increased BG expression. Note that the switch from M+ to M− (i.e., decrease in GB expression) actually occurred prior to the administration of exogenous insulin at POD 431. It was not until clear graft loss had occurred that the GB expression was once again elevated. This suggested that GB expression might be useful for staging of islet allograft and correlation of outcomes of other, cell-based assays with graft status.

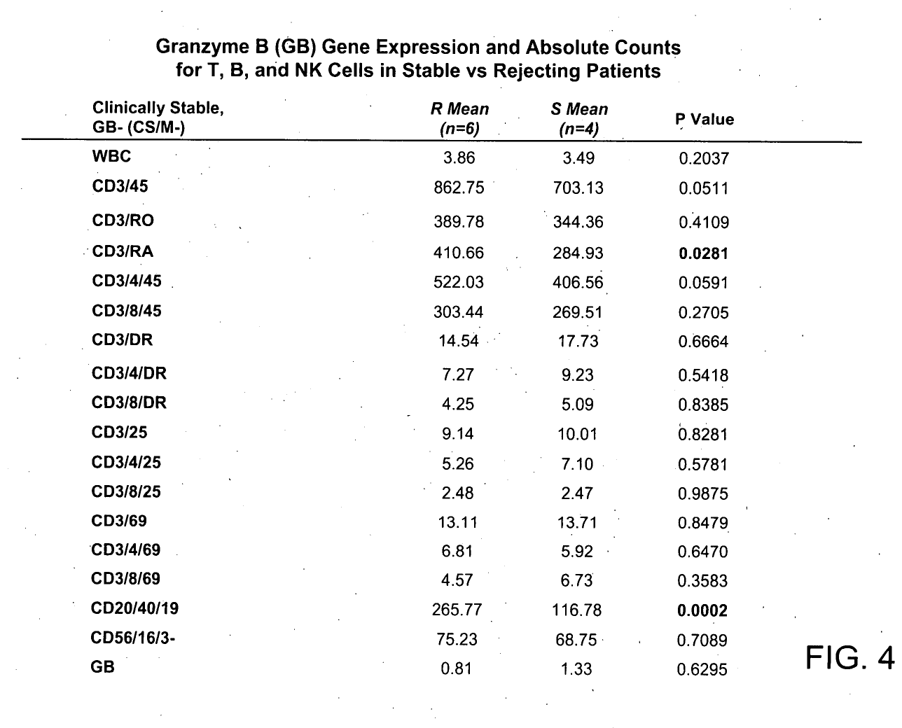

Prior to islet transplantation, patients were administered steroid-free immune suppression consisting of rapamycin (sirolimus), FK506 (tacrolimus), and an anti-IL2 receptor-specific monoclonal antibody (daclizumab). These and other agents such as dexamethasone or cyclosporine could be administered to suppress graft rejection or autoimmune disease. This drug regimen is known to result in lymphocyte depletion and both groups (R and S) experienced a drop in WBC and absolute lymphocyte subset counts (data analyzed after one month on immune suppression to allow the full suppressive effect to manifest). Although not statistically significant in FIG. 4, the WBC decreased by 45% for the S group and 31% for the R group of clinically stable patients for whom the molecular flag was not increased (CS/M−, the baseline was established post-transplant and one month after initiation of immune suppression). There was also no statistically significant difference in GB expression between R and S groups of patients. There was almost a statistically significant difference between R and S groups for the T-cell compartment (CD3+/CD45+) after one month of immune suppression (p=0.0511). The difference between R and S groups for the naïve CD3+/CD45RA+ T-cell subset was statistically significant (p=0.0281), as was the difference in the B-cell compartment (CD20+/CD40+/CD19+), with the R group retaining higher levels of these cells subsets as compared to the S group. Although both groups experienced decreases in absolute counts for all other subsets, the resultant difference between groups was not statistically significant for the other populations analyzed.

These data suggest that the initial effects of immune suppression on the T- and B-cell compartments may have an impact on whether or not a patient goes on to reject the transplant, with clinically stable patients experiencing significantly greater decreases in total T and B cells.

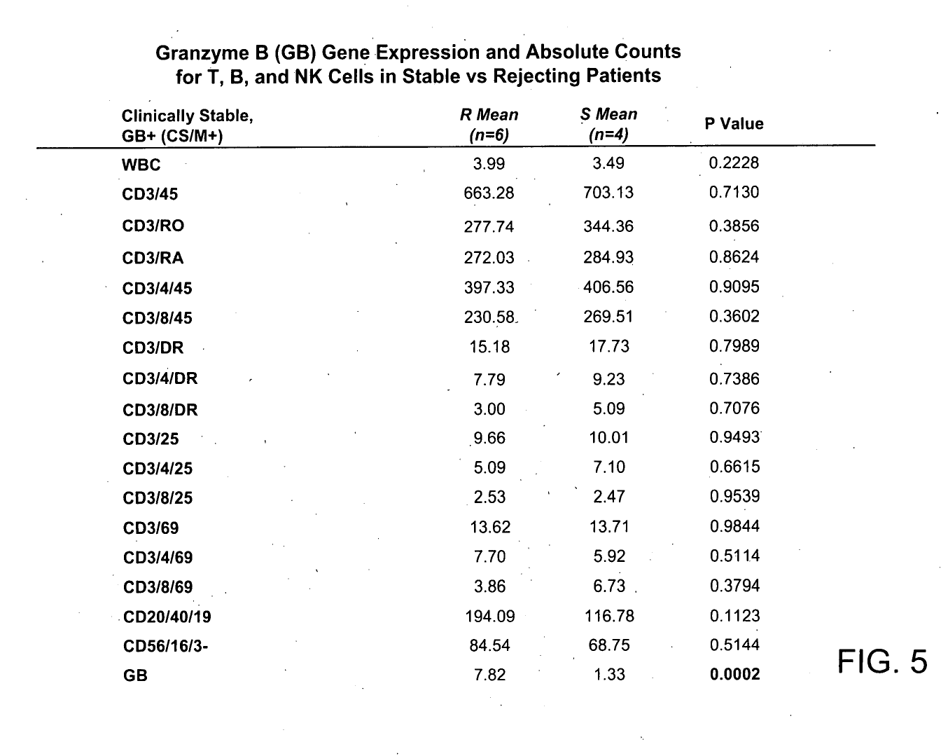

Using an increase in GB expression as a risk factor that indicated a propensity to activate immune recognition of alloantigen or self antigen, flow cytometry data for the R group was analyzed during periods of increased GB expression (FIG. 5). No significant differences were detected between R and S groups for any lymphocyte subset. These data demonstrate that, in the absence of an increase in expression of granzyme B, perforin, or Fas ligand, no significant differences between patients in the R and S groups would have been detected by flow cytometric analysis. Due to the relatively small number of time points routinely studied in clinical trials, changes in immunophenotype within the rejecting population would most likely have not been obvious in the setting of data from a single patient. Furthermore, since each rejecting patient experienced an increase in GB expression at a different time point after transplantation, the conventional practice of collecting samples from patients at predetermined times (e.g., every three months) would prevent detection of significant changes in immune status.

Frequent monitoring (no more than every 1-4 weeks, more frequently in the first six months post-transplant and decreasing in frequency thereafter) of expression (e.g., RNA transcription or protein translation) of granzyme B, perforin, or Fas ligand would enable detection of changes at the molecular level (e.g., expression profiling with a gene array, nuclease protection, primer extension, RT-PCR, immunoassay, mass spectroscopy, or nuclear magnetic resonance) that are routinely missed with cell-based studies such as flow cytometry, MLR, antigen-specific and/or HLA-specific stimulation, ELISPOT, etc. ELISA of separated cells and serum is not preferred to distinguish intracellular and secreted cytokine.

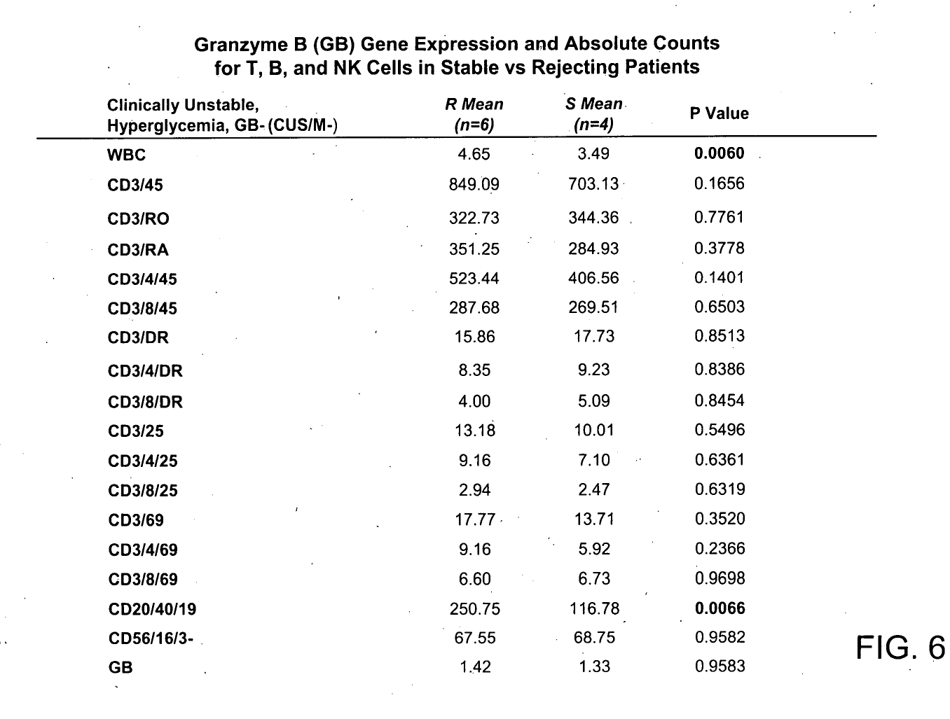

At the time of onset of clinical instability (e.g., hyperglycemia, initiation of exogenous insulin administration), an increase in GB expression was no longer detected in the R group (FIG. 6). Analysis of WBC in the R group during this time period revealed a further increase, resulting in a significant difference between patients that remained in the S and R groups. The difference in WBC in patients of the R group from the clinically stable patients without an increase in GB expression to occurrence of graft instability became significant.

Flow cytometry analyses revealed a statistically significant difference for the B-cell compartment (CD20+/CD40+/CD19+) between clinically unstable, GB− patients (CUS/M−) in the R and S groups, with significantly higher B cell numbers in the rejecting population. No other marker revealed a difference between the two groups. As previously mentioned, flow cytometric analyses of data from a single patient at a predetermined time point would not allow for detection of clinically relevant changes in phenotype. Without knowledge of this risk factor for the development of host vs. graft or autoimmune destruction of allogeneic or self (or host) tissue that occurred prior to the onset of clinical instability, there would be no indication of alterations in a subject's immune recognition of alloantigen or self antigen.

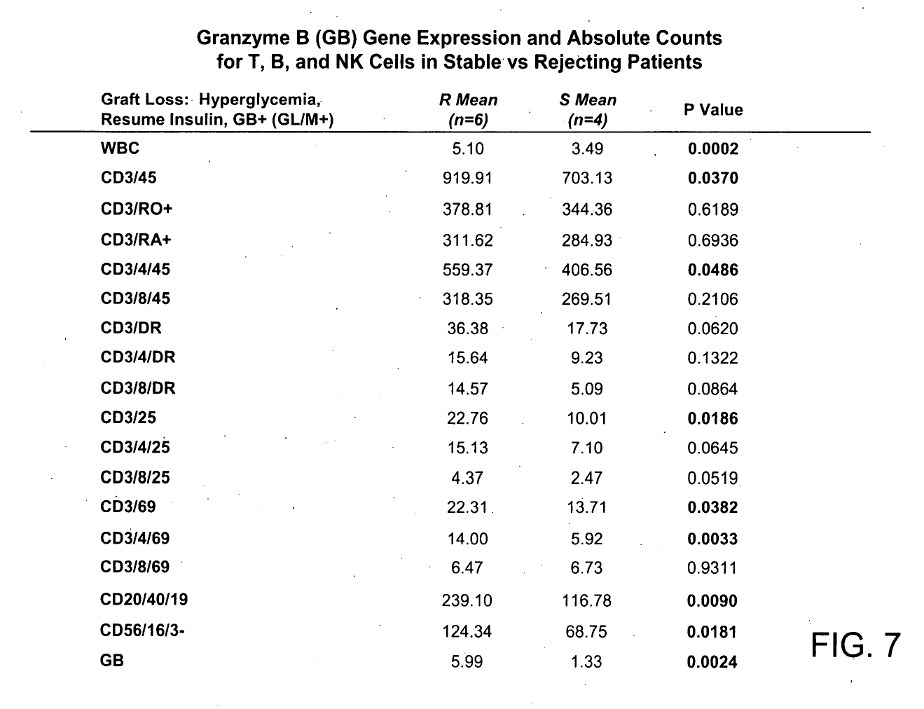

Once clear loss of functional islet mass has occurred, as indicated by frank hyperglycemia, increased need for exogenous insulin, and reduced production of C-peptide, several statistically significant differences were detectable. Hyperglycemic patients for whom substantial graft function was lost and the molecular flag was increased (GL/M+) showed a statistically significant difference in WBC between R and S groups of patients (FIG. 7). Also, statistically significant immunophenotypic differences were found between R and S patients for CD3+/CD45+ and CD3+/CD69+ T cell, CD20+/CD40+/CD19+ B cell, and NK cell (CD56+/CD16+/CD3−) counts (FIG. 7). Activated CD3+/CD25+ T cell counts were higher in patients of the R group, as were total (CD3+/CD4+/CD45+) and activated (CD3+/CD4+/CD69+) CD4+ T cell counts. GB expression was once again increased well above baseline in the R group and was significantly higher than in patients of the S group.

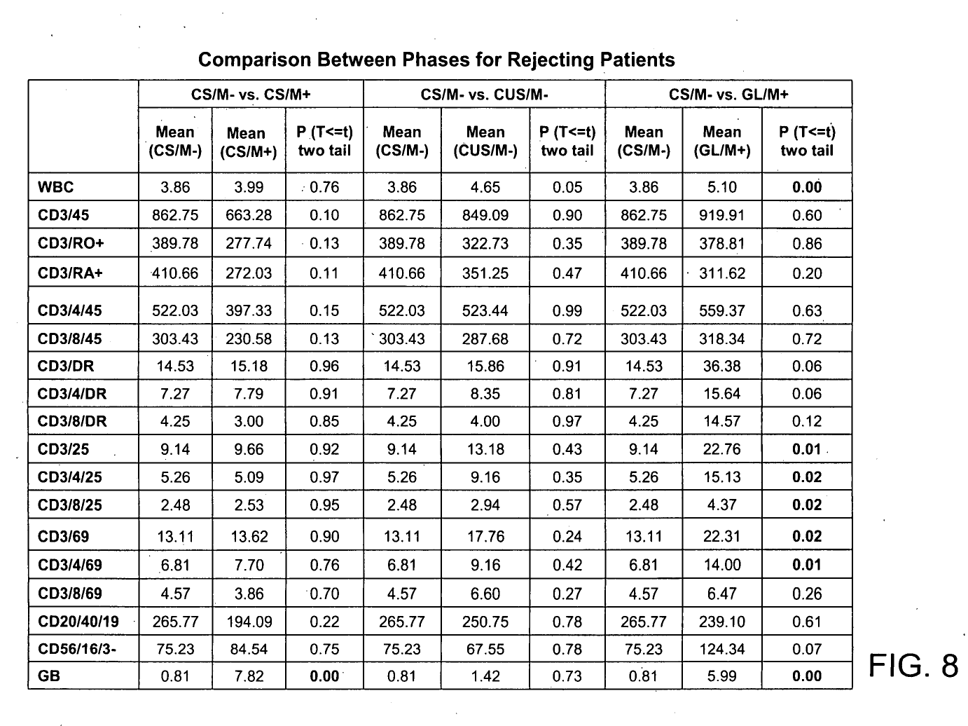

As compared to the absolute counts obtained for clinically stable patients of the R group without an increase in GB expression, the data for rejecting patients (those undergoing or who have undergone graft loss) reveal a statistically significant increase in WBC, activated CD4+ T cells (CD3+/CD4+/CD25+, CD3+/CD4+/CD69+), activated CD8+ T cells (CD3+/CD8+/CD25+) and GB expression (FIG. 8). It is possible during graft loss to observe significant changes in immunophenotype; at this point, however, it is too late to interfere in the immune mediated destruction of islet cells (either grafts or host cells depending on whether graft rejection or autoimmunity is involved).

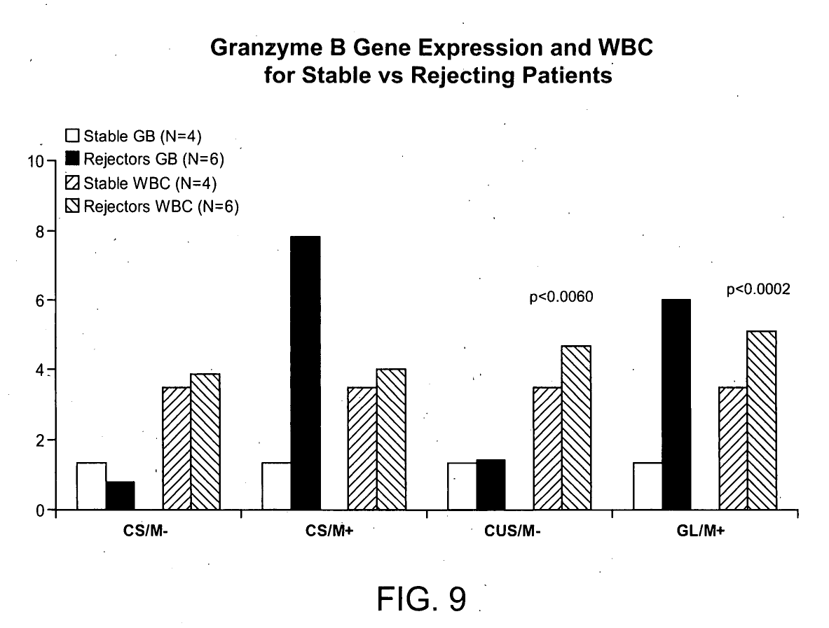

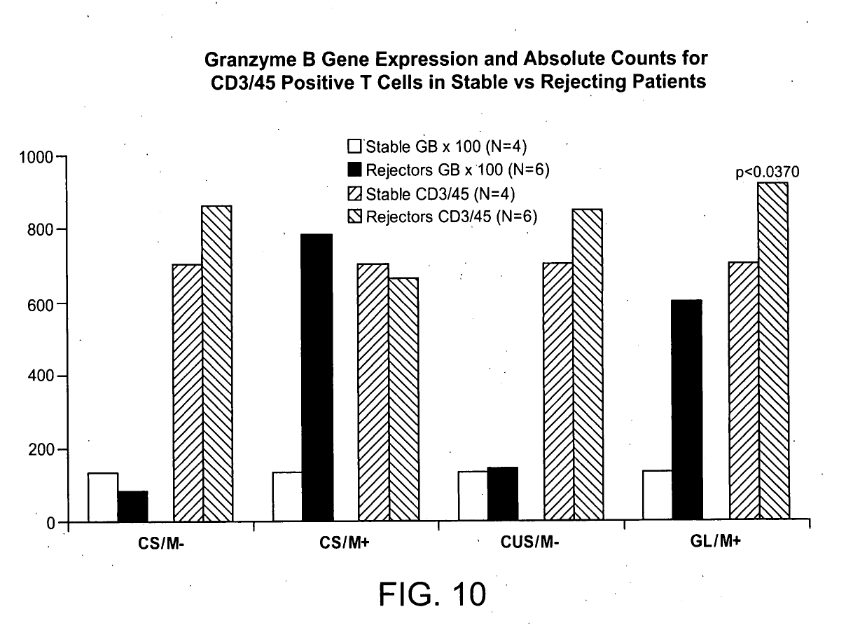

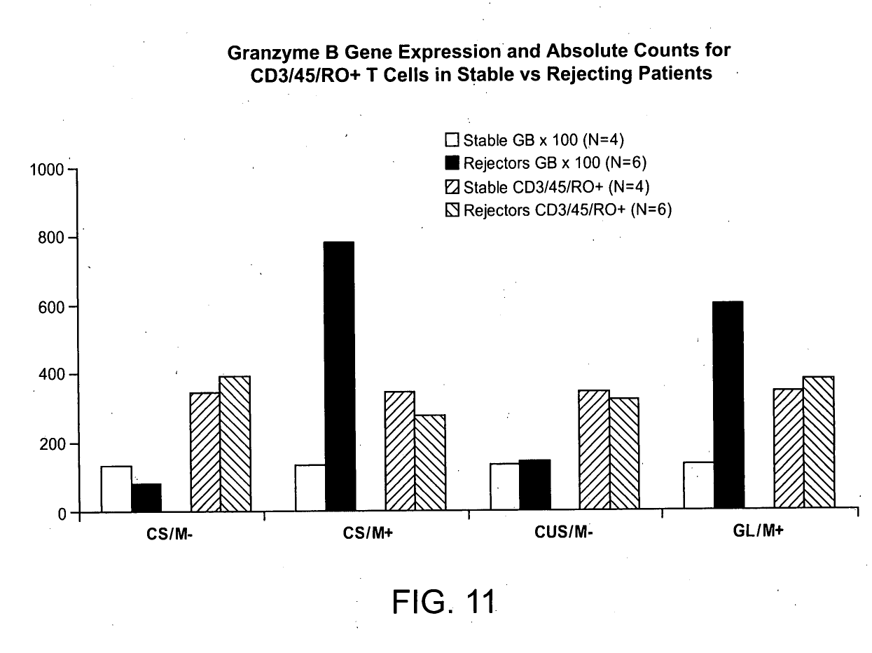

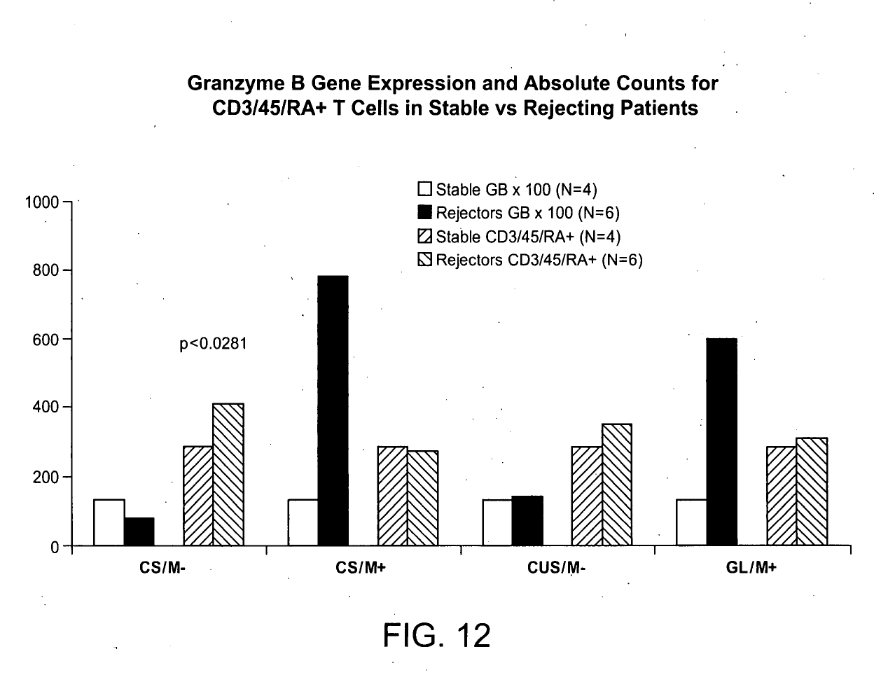

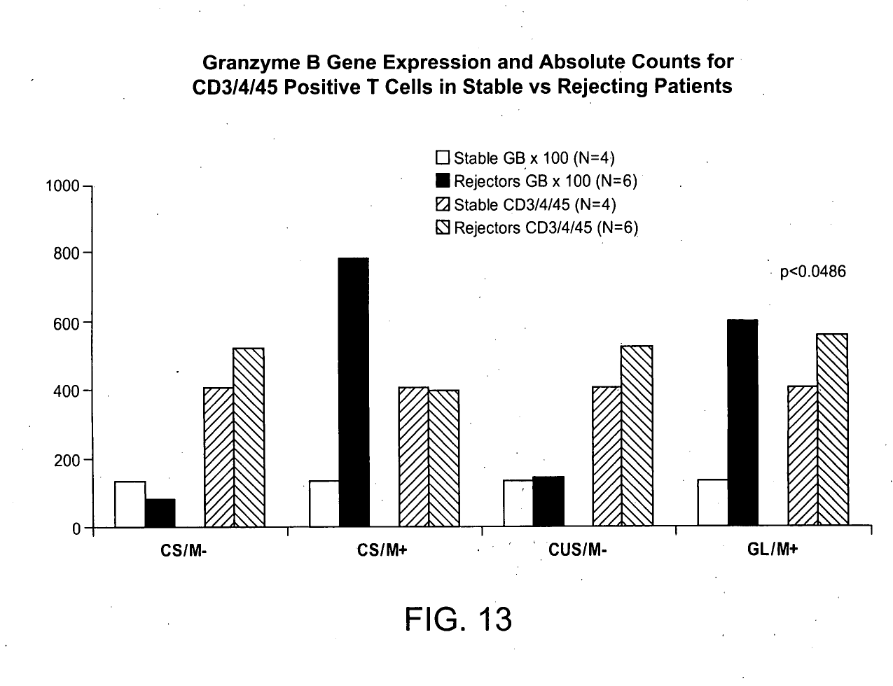

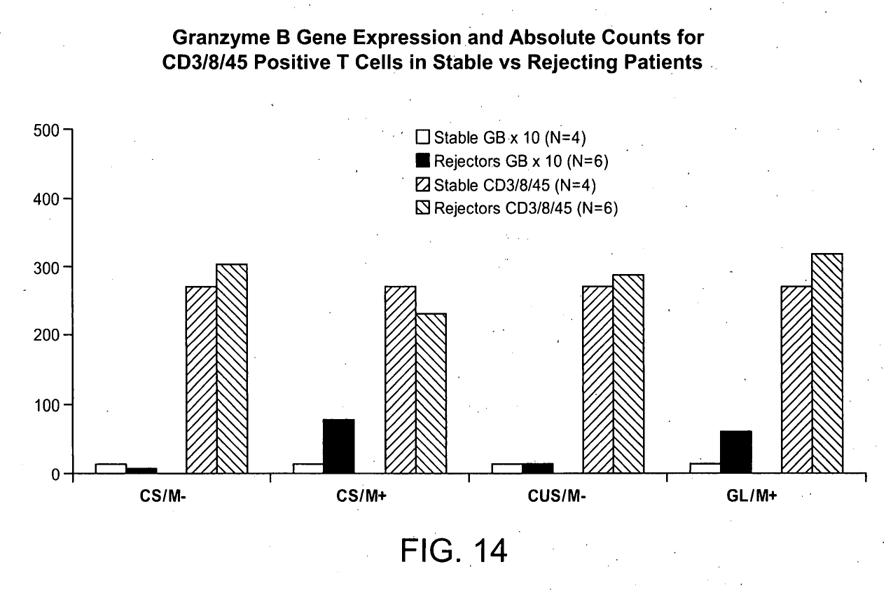

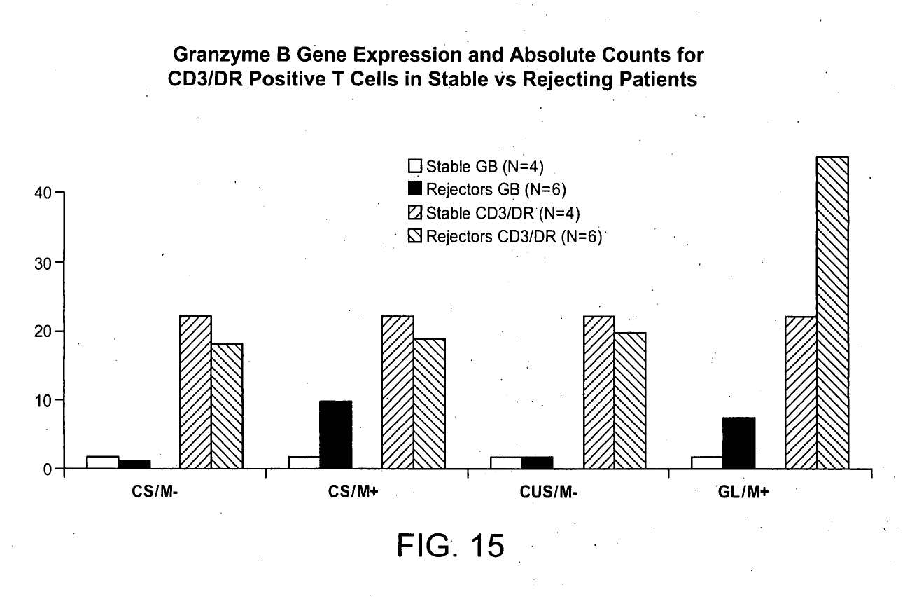

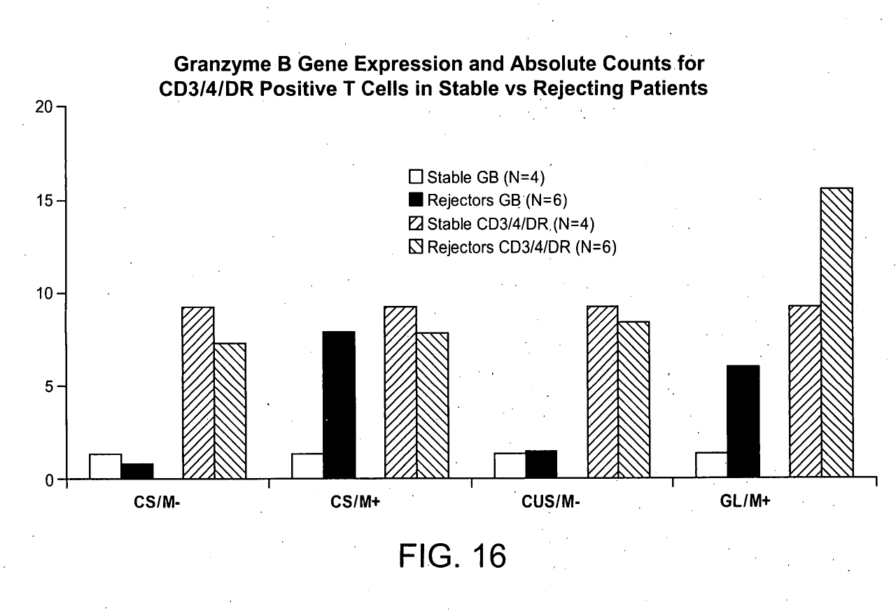

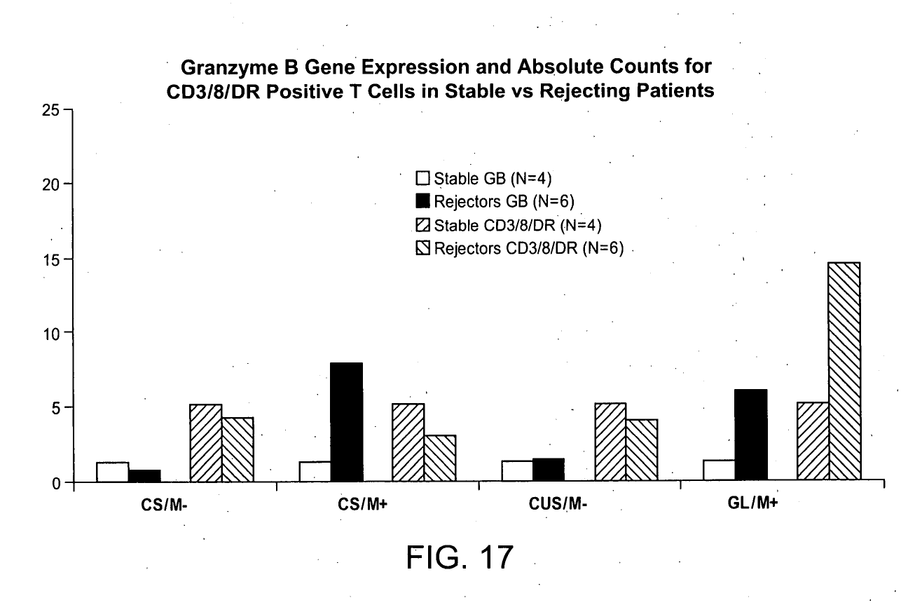

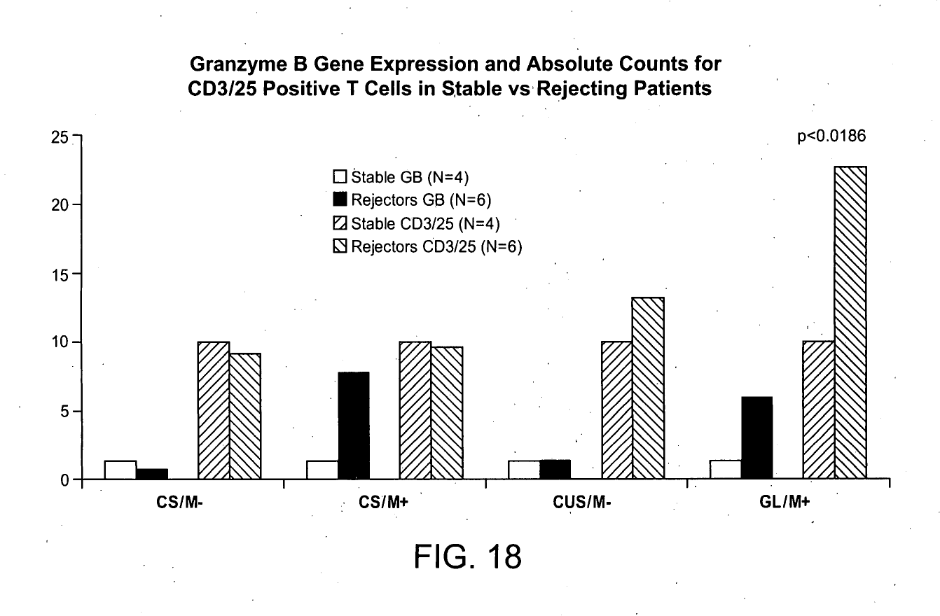

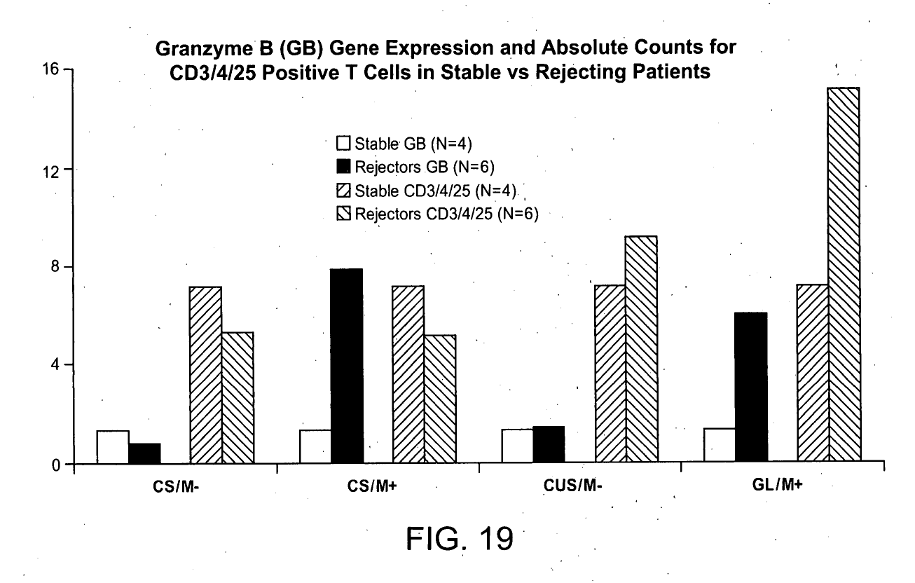

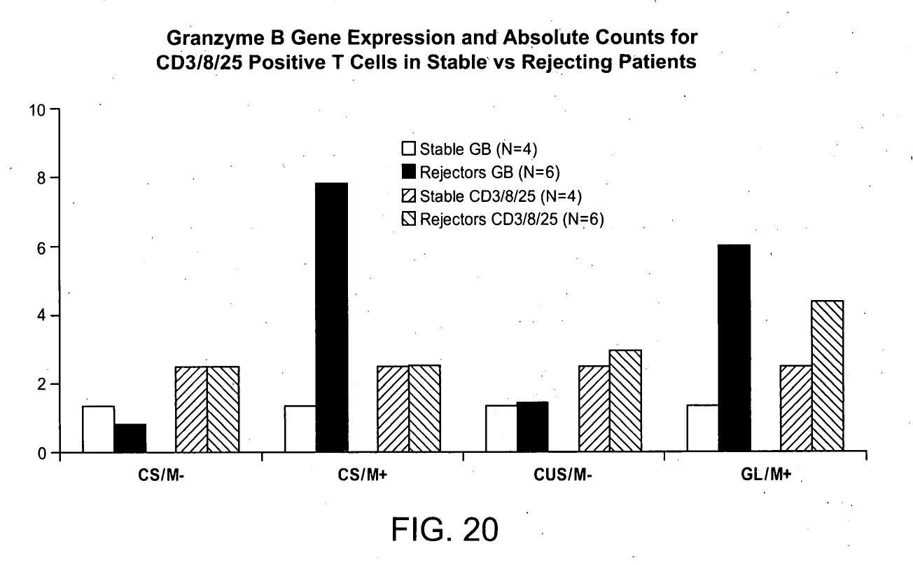

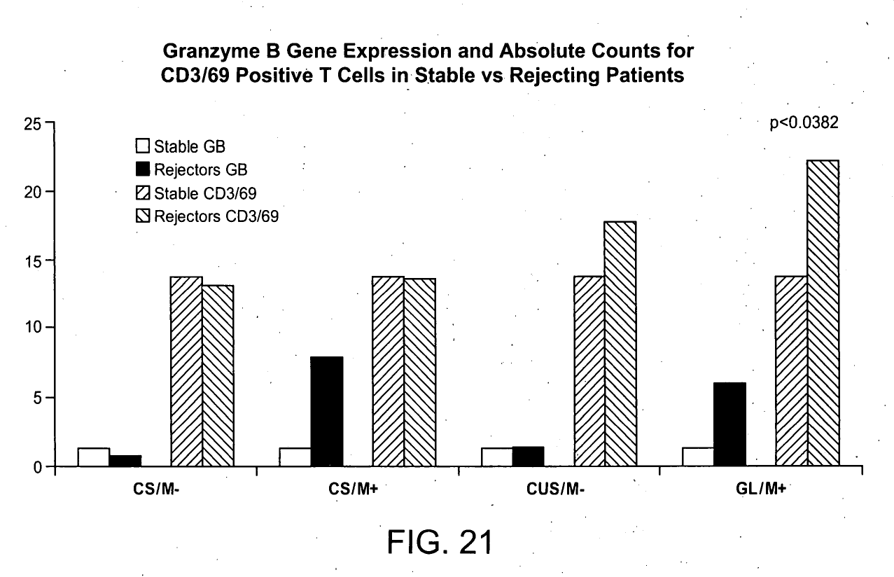

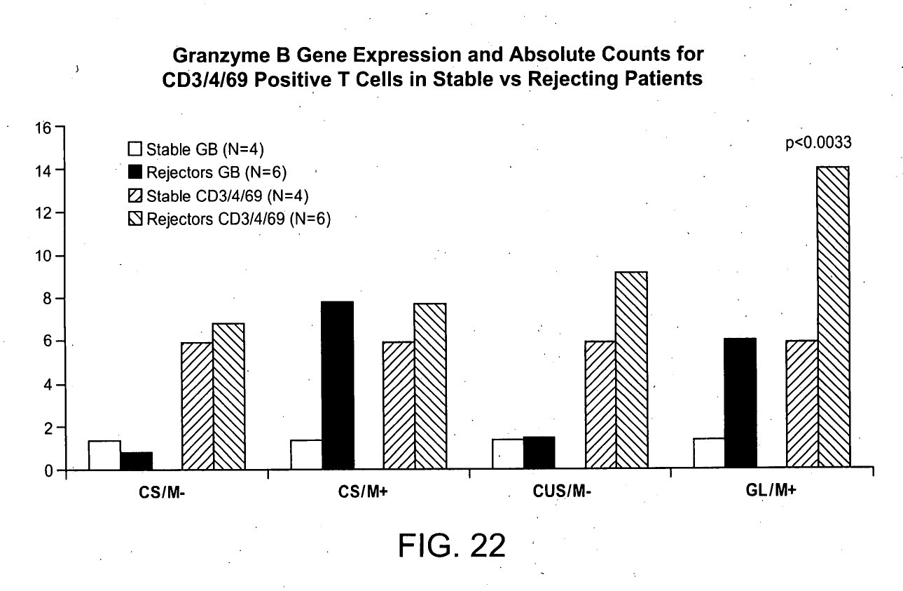

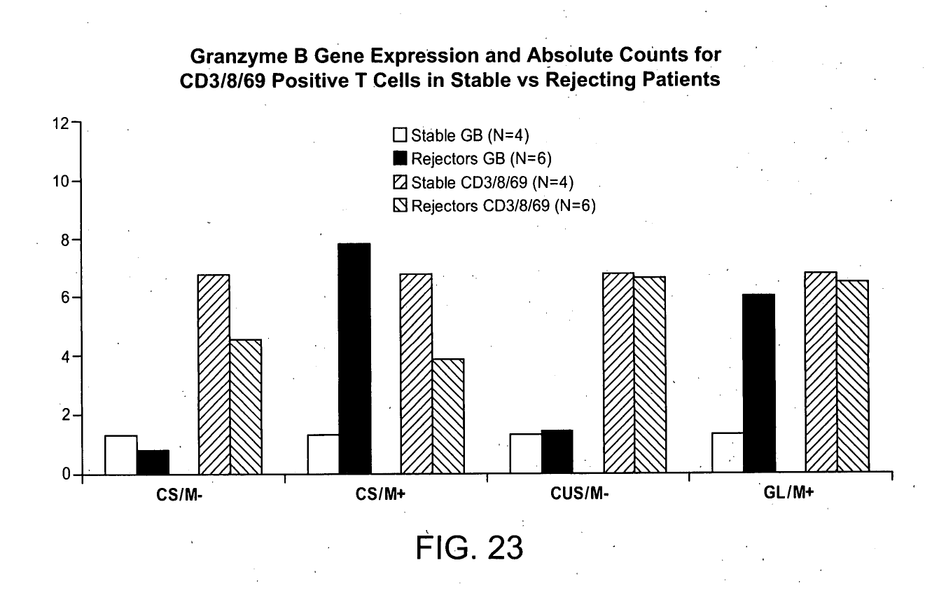

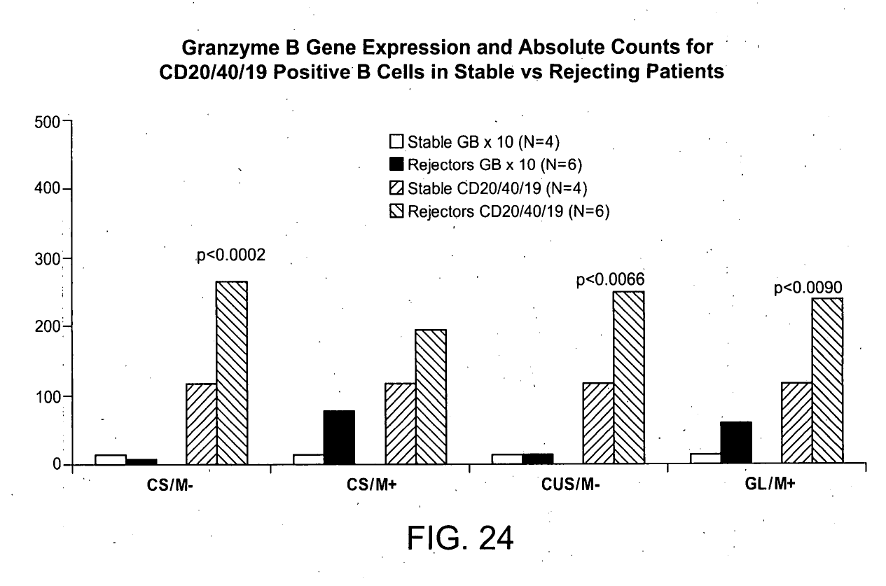

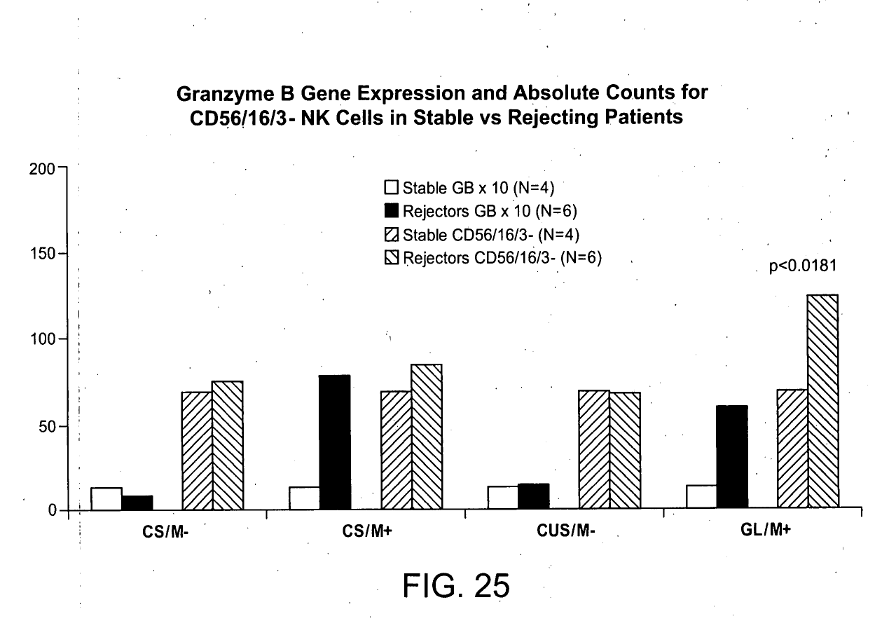

FIGS. 9-25 summarize the above results, provide further comparisons, and identify other statistically significant differences between patients of the R and S groups. The majority of significant cell surface changes detectable by flow cytometry in antigen nonstimulated cells occurred in the graft loss phase and the only clearly distinguishable feature prior to overt graft loss, is elevation of CLG expression (GB in this case).

Table 1 summarizes the statistical analyses of differences between R and S patient groups for granzyme B (GB) expression, the white blood cell count (WBC), lymphocyte subsets, and cellular assays of donor-specific responsiveness (i.e., mixed lymphocyte reaction or MLR) in the host as measured by proliferation of host lymphocytes when stimulated with irradiated donor PBMC. The anti-donor-specific MLR was depressed after initiation of steroid-free immune suppression (SFIS, this can vary with the immunosuppressive regimen), and the response remained suppressed during the clinically stable, GB+ phase. Return of the MLR begins during the CUS/M+ phase and is clearly positive again after graft loss. This supports the potential of molecular assays to detect rare event cells in the peripheral blood and to signal the investigator that it is time to obtain further blood samples for expansion of antigen-specific cells (in order to make them detectable at the cellular level) and determination of their functional (e.g., regulatory, effector) capacity.

TABLE 1
Summary of Statistical Analyses and MLR

| | GB | WBC | T | CD4 T | B | MLR |
|---|---|---|---|---|---|---|
| Pre-SFIS | 0.0129/ | NS | NS | NS | NS | + |
| | 0.6963 | | | | | |
| CS/M− | NS | NS | NS | NS | 0.0002 | +/Hypo |
| CS/M+ | 0.0002 | NS | NS | NS | NS | Hypo |
| CUS/M− | NS | 0.0060 | NS | NS | 0.0066 | Some+ |
| GL/M+ | 0.0024 | 0.0002 | 0.0370 | 0.0486 | 0.0090 | Most+ |

P less than 0.05 was considered a statistically significant difference

The immunophenotype data correlate with data obtained from analyses of anti-donor mixed lymphocyte cultures, in that all patients on this immunosuppressive regimen become nonreactive to donor cells over time. It is only after significant islet loss has occurred that anti-donor MLR reactivity reappears. This may be due in part to the use of predetermined time points that do not allow for detection of changes for each recipient (i.e., the immune change may have already occurred and was no longer detectable in peripheral blood).

Our molecular data suggest that activated T cells appear in the blood at a low level in the early phases of inflammation, then disappear at the time that graft instability is first noted. The activated T cells subsequently reappear after graft loss.

Currently, attempts to expand cells in order to allow for detection of rare event, antigen-specific T cells involve the use of nonspecific as well as antigen-specific stimulation. If a cell is present at low frequency, nonspecific stimulation could lead to dilution of the antigen-specific clone and allow for expansion of T cells that are not pertinent to the disease state or clinical condition. Our data support this possibility.

The inability to detect changes in immune status at the cellular level may be due to the fact that donor-specific T cells must be activated in regional lymph nodes, in a microenvironment that enables presentation of donor islet antigens. Activated T cells must then home to the transplantation site. The frequency of donor-specific or activated cells present in the peripheral blood at this time point will be very low (i.e., the donor-specific, activated T cells represent a rare event that is not easily detectable at the cellular level). Once the cells have homed to the graft, they are no longer detectable (at the molecular or cellular level) in peripheral blood. Subsequent to expansion of the activated donor and/or islet-specific T cells and destruction of tissue, it is once again possible to detect the cells in peripheral blood.

Our data demonstrate that activated, donor-specific and/or islet-specific T cells can be detected in peripheral blood early in the anti-graft response. Every patient responds to immune suppression in an individual manner and the time course to rejection will vary. Using an increase in the expression of granzyme B, perforin, or Fas ligand as a risk factor for activation of the host immune system, it may be possible to gain more information regarding the nature of the specific immune reaction (anti-donor=recipient vs. graft response; anti-islet=recurrence of autoimmune response) that is occurring and to intervene in a timely (and, ultimately, antigen specific) fashion. This would be done by using the increase in gene expression to signal the need to undertake antigen-specific T-cell assays that have the potential to distinguish between rejection and autoimmune disease. Increases in antigen-specific T-cell reactivity signal that the immune intervention is failing and additional measures need to be taken. Current practice in clinical trials is to establish time-consuming and large blood volume requiring assays on a 3- to 6-month, predetermined basis. Since antigen stimulation can lead to expansion of rare T-cell clones, such assays might enable detection of increasing antigen-specific immunoreactivity if they were undertaken at the critical time in which these cells actually are present in the peripheral blood, namely during migration of activated cells to the transplant site. Using predetermined time points, this window of opportunity may have passed. This is one of the reasons that it has been historically difficult to demonstrate that these assays have utility until the discover of the present invention. Furthermore, as demonstrated below, the use of nonspecific stimulation can dilute out the presence of rare event, antigen-specific T cells. It is preferred to undertake both nonspecific stimulation of cells to demonstrate that patients are not over immunosuppressed, as well as antigen-specific stimulation to identify the cells of interest.

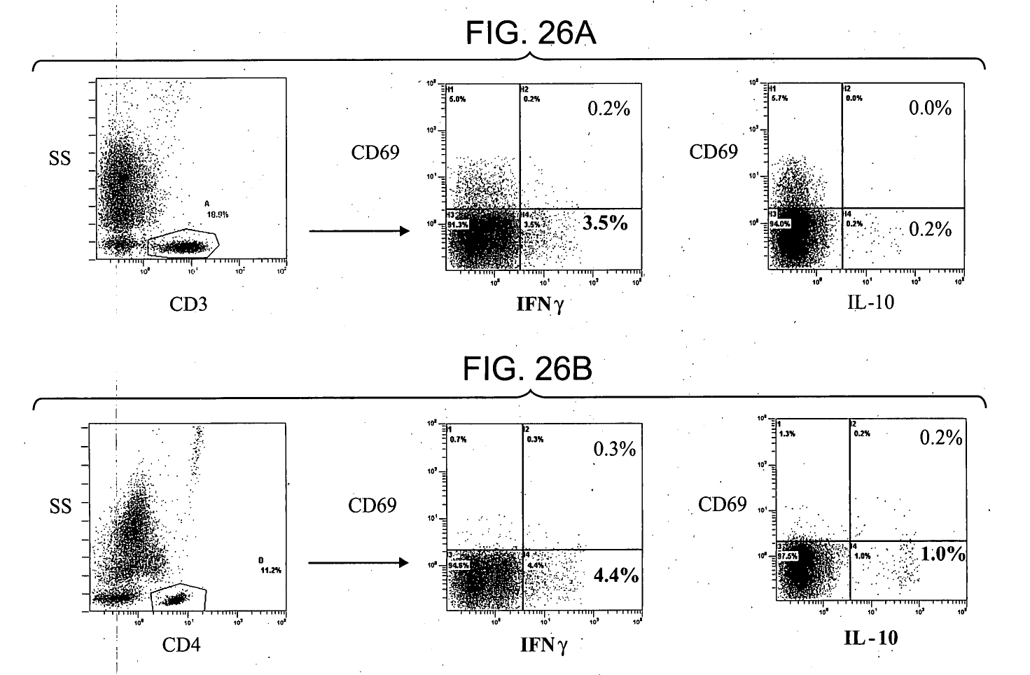

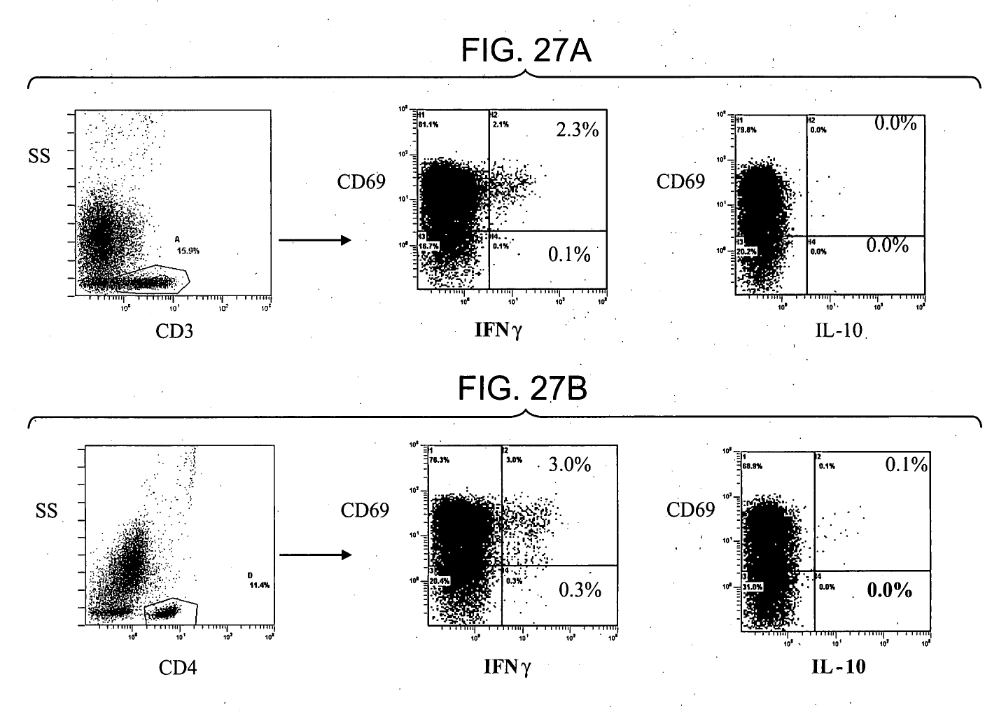

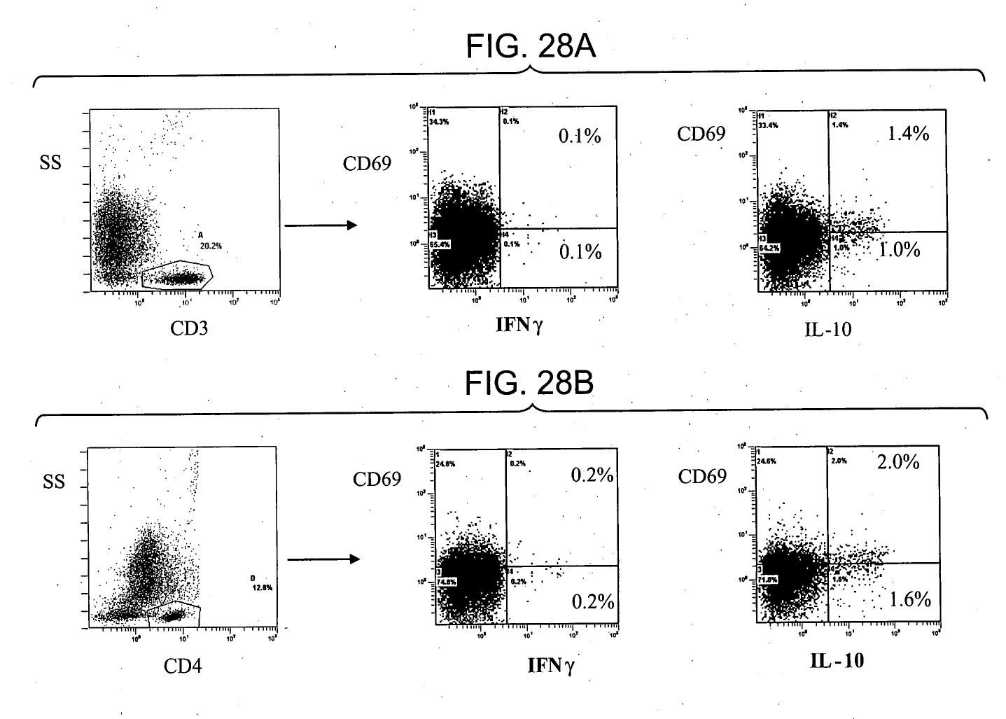

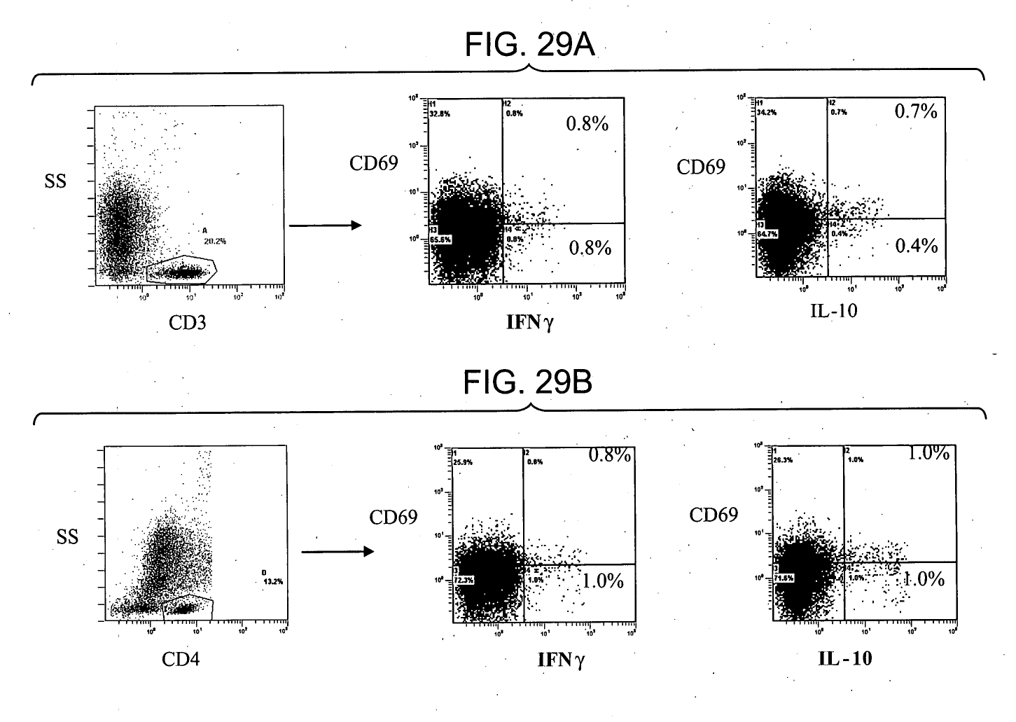

Intracellular cytokines associated with effector (inflammatory cytokine IFNγ) or regulatory (IL-10 cytokine) capabilities were detected in unstimulated PBMC (FIG. 26), specifically CD3+/CD69− and CD3+/CD4+/CD69− cells. After nonspecific stimulation with dextran crosslinked anti-CD3 plus anti-CD28 (i.e., a global activator), the IL-10+ population of cells was diluted out and no longer visible, while the IFNγ+ population of cells switched to CD69+ (i.e., CD3+/CD69+ and CD3+/CD4+/CD69+) (FIG. 27). In contrast, alloantigen-specific activation of the recipient's cells with PBMC from donor 1369 in an MLR (FIG. 28) increased the IL-10+ cells (regulatory phenotype), which are now mostly CD69+, and very few IFNγ+ cells (inflammatory phenotype) were detectable, thereby suggesting a regulatory balance for donor-specific T cells. Activation with PBMC from donor 1461 in an MLR (FIG. 29) increased the IL-10+ cells but also increased the IFNγ+ cells, thereby suggesting that the balance of immunity for donor-specific T cells favors rejection.

Table 2 summarizes these results and demonstrates how antigen-specific stimulation of T cell populations can provide information on the functional capacity of antigen-specific T cells obtained from a peripheral blood sample.

TABLE 2
Intracellular Cytokines

| | |
|---|---|
| % CD3+ | % CD4+ |

| Stimulus | IFN-γ | IL-10 | IFN-γ | IL-10 |
|---|---|---|---|---|
| Unstimulated | 3.8% | 0.2% | 5.1% | 1.2% |
| αCD3dex | 2.4% | 0.0% | 3.6% | 0.2% |
| αCD28 | | | | |
| Third Party | 4.2% | 1.1% | 5.4% | 2.5% |
| 1369 | 0.2% | 2.4% | 1.9% | 5.1% |
| 1461 | 1.5% | 1.1% | 4.3% | 2.6% |

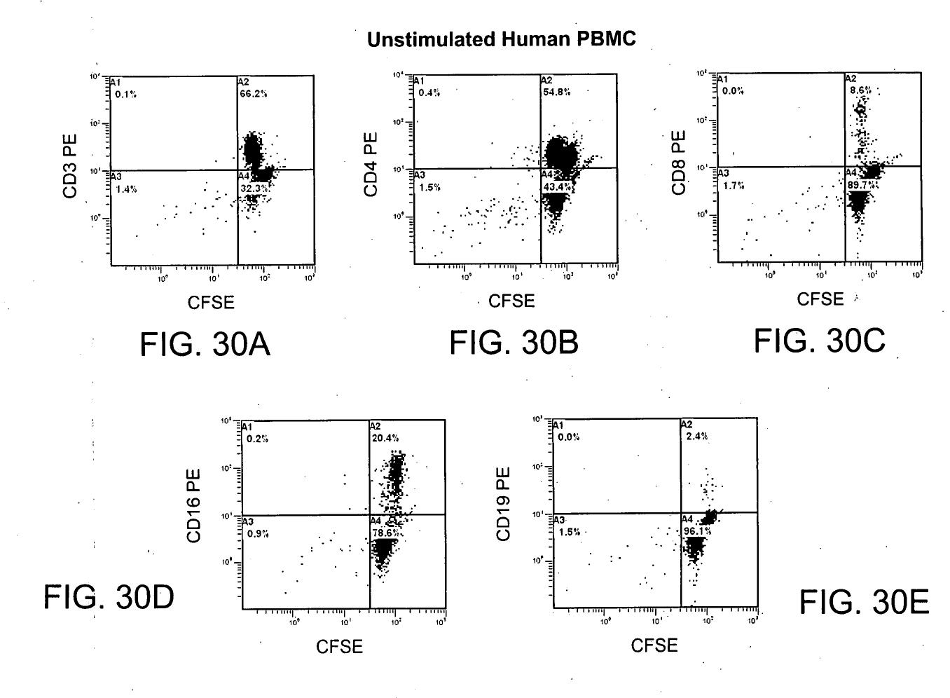

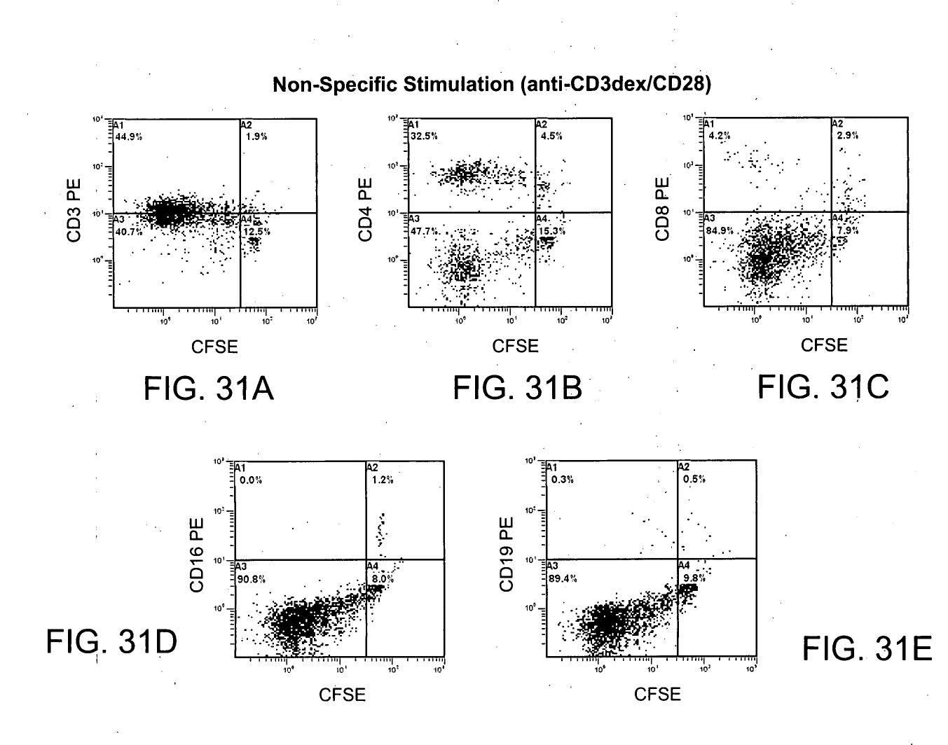

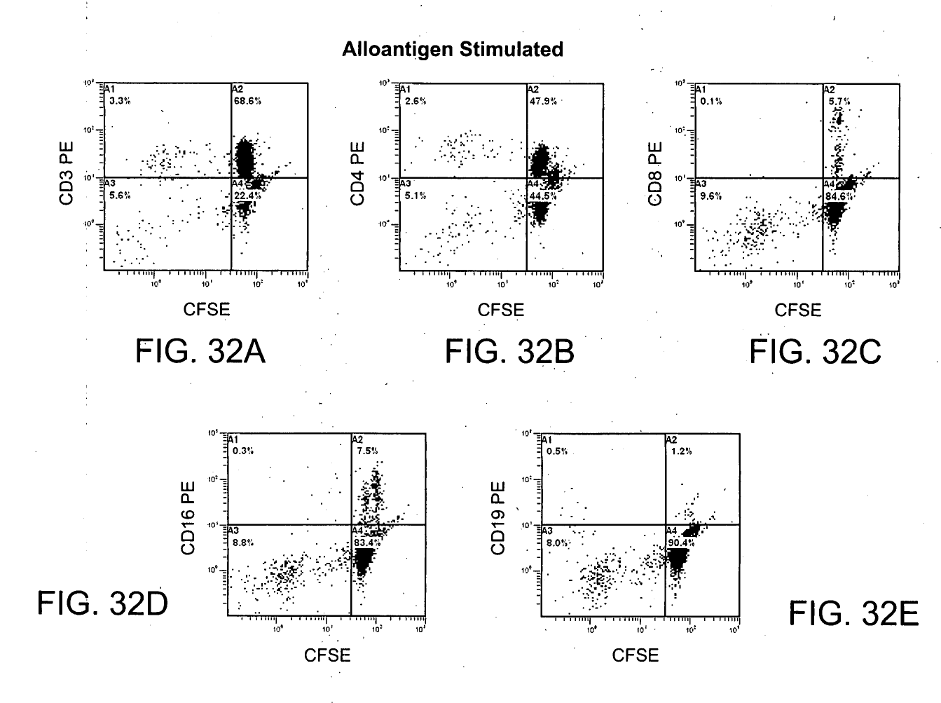

The differences in antigen-specific vs. nonspecific stimulation of cells is further demonstrated in FIGS. 30-32, in which the proliferation of recipient cells to donor cells was ascertained via flow cytometry (using CFSE dye incorporation and dilution with each cell division) for nonstimulated cells (FIG. 30), nonspecifically stimulated cells (FIG. 31), and alloantigen-stimulated cells (i.e., MLR, FIG. 32). Distinct differences in the degree of proliferation were observed as expected, and this method was combined with cell surface phenotype and analysis of intracellular cytokines. This allows gating on specific cell subpopulations that are responding to specific antigen stimulation (e.g., autoantigen, alloantigen of the donor or a third party, islet antigen of self) and determining their functional properties (e.g., expression of cytokines and their receptors, intracellular and cell surface activation of effector molecules, adhesion molecules, etc.). Utilization of a molecular flag to signal the presence of rare event cells in peripheral blood can allow for attainment of a sample volume sufficient in size to isolate PBMC for antigen-specific, functional assays.

Patents, patent applications, books, and other publications cited herein are incorporated by reference in their entirety.

All modifications and substitutions that come within the meaning of the claims and the range of their legal equivalents are to be embraced within their scope. A claim using the transition “comprising” allows the inclusion of other elements to be within the scope of the claim; the invention is also described by such claims using the transitional phrase “consisting essentially of” (i.e., allowing the inclusion of other elements to be within the scope of the claim if they do not materially affect operation of the invention) and the transition “consisting” (i.e., allowing only the elements listed in the claim other than impurities or inconsequential activities which are ordinarily associated with the invention) instead of the “comprising” term. Any of these three transitions can be used to claim the invention.

It should be understood that an element described in this specification should not be construed as a limitation of the claimed invention unless it is explicitly recited in the claims. Thus, the granted claims are the basis for determining the scope of legal protection instead of a limitation from the specification which is read into the claims. In contradistinction, the prior art is explicitly excluded from the invention to the extent of specific embodiments that would anticipate the claimed invention or destroy novelty. Moreover, no particular relationship between or among limitations of a claim is intended unless such relationship is explicitly recited in the claim. Similarly, all possible combinations and permutations of individual elements disclosed herein are considered to be aspects of the invention. Similarly, generalizations of the invention's description are considered to be part of the invention.

From the foregoing, it would be apparent to a person of skill in this art that the invention can be embodied in other specific forms without departing from its spirit or essential characteristics. The described embodiments should be considered only as illustrative, not restrictive, because the scope of the legal protection provided for the invention will be indicated by the appended claims rather than by this specification.

What is claimed is:

1. An in vitro method for monitoring development of an antigen-specific T-cell response to alloantigen, autoantigen, or self antigen against target cells of a subject, said method comprising:
(a) obtaining samples from the subject,
(b) determining whether expression of at least one of the group consisting of granzyme B, perforin, and Fas ligand is increased in samples from the subject, and
(c) determining whether T cells recognizing alloantigen, autoantigen, or self antigen are present in samples from the subject by a functional assay after expression of at least one of granzyme B, perforin, and Fas ligand is increased.

2. The method of claim 1, wherein at least one of the samples is obtained from the subject before immunosuppressive treatment to establish a baseline for expression.

3. The method of claim 1, wherein at least one of the samples is obtained from the subject before an increase in expression is detected.

4. The method of claim 1, wherein each sample is less than 10 mL in volume.

5. The method of claim 1, wherein samples are obtained from the subject at least every two months.

6. The method of claim 1, wherein an initial sample is obtained from the subject within 24 hours of transplantation.

7. The method of claim 1, wherein RNA transcription is measured to determine levels of expression.

8. The method of claim 1, wherein protein translation is measured to determine levels of expression.

9. The method of claim 1 further comprising isolating mononuclear cells of the peripheral blood to be functionally assayed.

10. The method of claim 1, wherein increased expression is determined before substantial destruction of target cells causes a physiological change in the subject.

11. The method of claim 1, wherein the antigen is alloantigen, autoantigen, or self antigen.

12. The method of claim 1, wherein the functional assay is selected from the group consisting of cell proliferation, cytokine production, ELISPOT, immunophenotyping, limiting dilution analysis, mixed lymphocyte reaction, and tetramer technology.

13. The method of claim 1, wherein the functional assay determines whether or not T cells which are restricted by the subject's major histocompatibility complex (MHC) are present.

14. The method of claim 1, wherein the functional assay determines whether or not T cells which recognize alloantigen, autoantigen, or self antigen are present.

15. The method of claim 1, wherein the functional assay determines whether or not activated T cells are present.

16. The method of claim 1, wherein the functional assay determines whether either alloantigen, autoantigen, or self antigen is recognized by activated T cells.

17. The method of claim 1, wherein the functional assay determines whether or not regulatory and/or inflammatory cytokines or chemokines are produced by T cells.

18. The method of claim 1, wherein the functional assay is comprised of interacting human leukocyte antigens (HLA) of donor or host with at least cells of the sample.

19. The method of claim 1, wherein the functional assay is comprised of interacting alloantigen, autoantigen, or self antigen with at least cells of the sample.

20. The method of claim 1, wherein the functional assay measures frequency of lymphocytes which recognize alloantigen, autoantigen, or self antigen in the sample.

21. The method of claim 1, wherein the subject has been transplanted with a solid organ, tissue, or cells thereof.

22. The method of claim 1, wherein the subject has been transplanted with stem cells.

23. The method of claim 1 further comprising inhibiting development of the antigen-specific T-cell response after (b) increased expression and (c) the presence of T cells recognizing alloantigen, autoantigen, or self antigen are determined.

24. The method of claim 23, wherein one or more steroids, macrolides, cytotoxic antibodies, or any combination thereof is administered to the patient to suppress the antigen-specific T-cell response.

25. A kit for monitoring development of an antigen-specific T-cell response to alloantigen, autoantigen, or self antigen against target cells of a subject, said kit comprising in one or more containers: (i) reagents to determine CLG expression; (ii) assay reagents to determine whether T cells recognize alloantigen, autoantigen, or self antigen by their function; (iii) reagents for cell culture; and (iv) reagents for cell analysis.
